(19) United States
(12) Patent Application Publication
(10) Pub. No.: US 2025/0332196 A1
(43) Pub. Date: Oct. 30, 2025

(54) ANTI-CLL-1 CHIMERIC ANTIGEN RECEPTORS, ENGINEERED CELLS AND RELATED METHODS

(71) Applicant: Caribou Biosciences, Inc., Berkeley, CA (US)

(72) Inventors: Leslie Edwards, San Ramon, CA (US); Steven B. Kanner, Los Angeles, CA (US); Erin K. Kelly, Berkeley, CA (US); Sai Valli Srujana Namburi, Richmond, CA (US)

(73) Assignee: Caribou Biosciences, Inc., Berkeley, CA (US)

(21) Appl. No.: 18/842,608

(22) PCT Filed: Nov. 13, 2023
(86) PCT No.: PCT/US2023/079508

§ 371 Date: Aug. 29, 2024

Related U.S. Application Data
(60) Provisional application No. 63/383,654, filed on Nov. 14, 2022.

(51) Int. Cl. A61K 35/17; A61K 40/11; A61K 40/31; A61K 40/42; A61P 35/02; C07K 14/705; C07K 14/725; C07K 16/28; C12N 5/0783; C12N 9/22; C12N 15/11; G01N 33/574

(52) U.S. Cl. CPC A61K 35/17; A61K 40/11; A61K 40/31; A61K 40/42; A61P 35/02; C07K 14/7051; C07K 14/70517; C07K 14/70521; C07K 16/2851; C12N 5/0636; C12N 9/226; C12N 15/111; G01N 33/57426; G01N 33/57492; A61K 2239/13; A61K 2239/17; A61K 2239/21; A61K 2239/48; C07K 2317/622; C12N 2310/20; G01N 2333/7056

(57) ABSTRACT

An anti-CD371 (anti-CLL-1) chimeric antigen receptor (CAR), engineered immune cells comprising the CAR, as well as therapeutic compositions, therapeutic methods and companion diagnostic methods are disclosed herein.

### CROSS-REFERENCE TO RELATED APPLICATIONS

This application claims priority to the U.S. provisional application Ser. No. 63/383,654, filed on Nov. 14, 2022.

### FIELD OF THE INVENTION

The invention related to the field of oncology and more specifically, to cell therapy with genetically engineered tumor-targeting immune cells.

### STATEMENT REGARDING FEDERALLY SPONSORED RESEARCH

None.

### BACKGROUND OF THE INVENTION

The American Cancer Society reports that acute myeloid leukemia (AML) accounts for about ⅓ of annual cases of leukemia in the United States but is responsible for nearly ½ of leukemia-related deaths. AML continues to inflict a severe death toll on both senior and younger patients. AML is associated with low 1-year survival rates in older patients and high relapse rates in younger patients who have achieved a remission. Traditional AML treatment is aggressive chemotherapy. A more innovative US FDA-approved treatment involves a combination of a Bcl-2 inhibitor (venetoclax) and a hypomethylating agent azacytidine (Ven/aza). Ven/aza treatment has a non-response rate of 30%. Small molecule inhibitors targeting FLT3, IDH and Hedgehog pathway proteins are currently investigational AML treatments. Mylotarg™ targeting CD33 is the first antibody-drug conjugate (ADC) currently approved in the U.S. and Japan for the treatment of AML but only in older and relapsed patients no longer eligible for chemotherapy.

There is a need for innovative treatments for AML that are associated with higher response and cure rates than the existing therapeutic modalities.

### SUMMARY OF THE INVENTION

In some embodiments, the invention is a chimeric antigen receptor (CAR) comprising: an anti-CD371 (anti-CLL-1) scFv; a transmembrane domain; a co-stimulatory domain; and a CD3 zeta domain. In some embodiments, the CAR further comprises a hinge domain. In some embodiments, the anti-CD371 (anti-CLL-1) scFv is represented by a formula V_{H}-Lₙ-V_{L} or V_{L}-Lₙ-V_{H}, wherein V_{H} comprises SEQ ID NO: 7, V_{L} comprises SEQ ID NO: 11, L is a peptide linker, and n is an integer between 1 and 5. In some embodiments, the peptide linker is represented by a formula (GₓS_{y})ₙ, wherein G is glycine, S is serine, and x, y, and n independently are integers between 1 and 5 (SEQ ID NO: 42), e.g., the linker comprises SEQ ID NO: 1 or SEQ ID NO: 2. In some embodiments, the anti-CD371 (anti-CLL-1) scFv comprises SEQ ID NO: 5. In some embodiments, the anti-CD371 (anti-CLL-1) scFv consists essentially of SEQ ID NO: 5. In some embodiments, the anti-CD371 scFv comprises SEQ ID NO: 3. In some embodiments, the anti-CD371(anti-CLL-1) scFv consists essentially of SEQ ID NO: 3. In some embodiments, the anti-CD371 (anti-CLL-1) scFv comprises SEQ ID NO: 4. In some embodiments, the anti-CD371 (anti-CLL-1) scFv consists essentially of SEQ ID NO: 4. In some embodiments, the anti-CD371 (anti-CLL-1) scFv comprises SEQ ID NO: 6. In some embodiments, the anti-CD371 (anti-CLL-1) scFv consists essentially of SEQ ID NO: 6. In some embodiments, the cytoplasmic domain comprises a CD28 co-stimulatory domain. In some embodiments, the cytoplasmic domain further comprises a CD3zeta domain. In some embodiments, the transmembrane domain comprises a CD8 transmembrane domain. In some embodiments, the CD8 transmembrane domain consists essentially of SEQ ID NO: 16. In some embodiments, the hinge domain comprises a CD8 hinge domain. In some embodiments, the CD8 hinge domain consists essentially of SEQ ID NO. 15. In some embodiments, the hinge domain comprises a CD28 hinge domain. In some embodiments, the hinge domain consists essentially of the CD28 hinge domain. In some embodiments, the CAR further comprises a signal peptide. In some embodiments, the signal peptide comprises a CD28 signal peptide.

In some embodiments, the CAR comprises a sequence selected from SEQ ID NOs.: 18, 19, 20, 21, 22, 23, 24, 25, and 26. In some embodiments, the CAR consists essentially of a sequence selected from SEQ ID Nos.: 18, 19, 20, 21, 22, 23, 24, 25, and 26. In some embodiments, the CAR is encoded by a sequence selected from SEQ ID NOs: 28, 29, 30, 31, 32, 33, 34, and 35.

In some embodiments, the invention is an isolated nucleic acid comprising a vector sequence and a sequence encoding the chimeric antigen receptor (CAR) described herein. In some embodiments, isolated nucleic acid further comprises a promoter selected from the group consisting of PGK1 promoter, MND promoter, Ubc promoter, CAG promoter, CaMKIIa promoter, SV40 early promoter, SV40 late promoter, the cytomegalovirus (CMV) immediate early promoter, Rous sarcoma virus long terminal repeat (RSV-LTR) promoter, mouse mammary tumor virus long terminal repeat (MMTV-LTR) promoter, β-interferon promoter, the hsp70 promoter EF-1α promoter, and 3-Actin promoter. In some embodiments, the vector comprises a plasmid. In some embodiments, the vector comprises a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV).

In some embodiments, the isolated nucleic acid comprises a sequence selected from SEQ ID NOs: 28, 29, 30, 31, 32, 33, 34, and 35.

In some embodiments, the invention is an immune cell comprising the chimeric antigen receptor (CAR) described herein. In some embodiments, the immune cell is selected from cells consisting of a T-cell and precursors thereof. In some embodiments, the T cell is selected from the group consisting of a T-helper cell, a cytotoxic T cell, and a regulatory T cell. In some embodiments, the CAR comprises a sequence selected from SEQ ID NO: selected from 18, 19, 20, 21, 22, 23, 24, 25, and 26. In some embodiments, the CAR comprises a sequence selected from SEQ ID NOs.: 18, 19, 20, 21, 22, 23, 24, 25, and 26. In some embodiments, the CAR is inserted into the T-cell receptor alpha chain (TRAC) locus. In some embodiments, the CAR is inserted into the TRAC locus on human chromosome 14 between nucleotides 22547538 and 22547539.

In some embodiments, immune cell of further comprises an armoring genomic modification. In some embodiments, the armoring genomic modification comprises inactivation of an immune checkpoint gene selected from the group consisting of PDCD1, CTLA-4, LAG3, Tim3, BTLA, BY55, TIGIT, B7H5, LAIR1, SIGLEC10, and 2B4. In some embodiments, the armoring genomic modification comprises an inactivation of the PDCDJ gene and the PDCD1 gene is cleaved between nucleotides 241852860 and 241852883. In some embodiments, the armoring genomic modification comprises inactivation of the beta-2 microglobulin (B2M) gene. In some embodiments, the armoring genomic modification comprises insertion of an HLA-E-B2M fusion coding sequence. In some embodiments, the HLA-E-B2M fusion coding sequence is inserted into the B2M locus. In some embodiments, the HLA-E-B2M fusion coding sequence is inserted into the B2M locus on human chromosome 15 between nucleotides 44715624 and 44715625. In some embodiments, the armoring genomic modification comprises an inactivation of the PDCD1 gene and an insertion of an HLA-E-B2M fusion coding sequence into the B2M gene.

In some embodiments, the invention is a method of making the immune cell described herein, the method comprising introducing into a cell a nucleic acid comprising a sequence selected from SEQ ID NOs.: 27, 28, 29, 30, 31, 32, 33, 34, and 35, and a nucleic acid encoding SEQ ID NO.: 40 and further comprising disrupting the PDCDJ gene in the cell. In some embodiments, the cell selected from cells consisting of a T-cell and precursors thereof.

In some embodiments, the introducing step comprises introducing into the cell a sequence-dependent endonuclease. In some embodiments, the introducing step comprises introducing into the cell a CRISPR system comprising a nucleic acid-guided endonuclease and nucleic acid-targeting nucleic acid (NATNA) guides. In some embodiments, the nucleic acid-guided endonuclease is selected from Cas9, Casl2a and CASCADE. In some embodiments, one or more components of the CRISPR system are introduced into the cell in the form of DNA. In some embodiments, the one or more components of the CRISPR system are introduced into the cell in the form of RNA. In some embodiments, the CRISPR system is introduced into the cell in the form of a nucleoprotein complex.

In some embodiments, the endonuclease comprises a catalytically inactive CRISPR endonuclease conjugated to the cleavage domain of the restriction endonuclease Fok I. In some embodiments, the endonuclease is selected from the group consisting of a zinc finger nuclease (ZFN), a ZFN-Fok I fusion, a transcription activator-like effector nuclease (TALEN), and a TALEN-Fok I fusion.

In some embodiments, the endonuclease cleaves the genome of the cell at a locus selected from the group consisting of TRAC, CBLB, PDCD1, CTLA-4, LAG3, Tim3, BTLA, BY55, TIGIT, B7H5, LAIR1, SIGLEC10, 2B4, and B2M. In some embodiments, the endonuclease cleaves the TRAC locus on human chromosome 14 between nucleotides 22547538 and 22547539.

In some embodiments, the endonuclease forms a nucleoprotein complex with a guide nucleic acid comprising a targeting region having SEQ ID NO.: 37. In some embodiments, a CAR-encoding nucleic acid comprising a sequence selected from 27, 28, 29, 30, 31, 32, 33, 34, and 35 is inserted into the cleaved TRAC locus. In some embodiments, the endonuclease cleaves the B2M locus. In some embodiments, the endonuclease cleaves the B2M locus on human chromosome 15 between nucleotides 44715624 and 44715625. In some embodiments, the endonuclease forms a nucleoprotein complex with a guide nucleic acid comprising a targeting region having SEQ ID NO.: 38. In some embodiments, a sequence encoding the HLA-E-B2M fusion of SEQ ID NO.: 40 is inserted into the cleaved B2Mlocus. In some embodiments, the CAR-encoding nucleic acid and the HLA-E-B2M encoding nucleic acid are introduced into the cell via electroporation. In some embodiments, the CAR-encoding nucleic acid and the HLA-E-B2M encoding nucleic acid are introduced into the cell via electroporation of naked DNA. In some embodiments, the CAR-encoding nucleic acid and the HLA-E-B2M encoding nucleic acid are introduced into the cell via a vector. In some embodiments, the vector is a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV). In some embodiments, disrupting of the PDCD1 gene comprises introducing into the cell a CRISPR Cas12 endonuclease and a guide nucleic acid comprising SEQ ID NO.: 39. In some embodiments, the endonuclease cleaves the PDCD1 locus on human chromosome 2 between nucleotides 241852860 and 241852883.

In some embodiments, the invention is a composition comprising the immune cell described herein and a pharmaceutically acceptable excipient. In some embodiments, the pharmaceutically acceptable excipient comprises one or more of carbohydrates, inorganic salts, antimicrobial agents, antioxidants, surfactants, buffers, acids, bases, water, alcohols, polyols, glycerin, vegetable oils, phospholipids, surfactants, sugars, derivatized sugars, alditols, mannitol, xylitol, maltitol, lactitol, xylitol, sorbitol, pyranosyl sorbitol, myoinositol, aldonic acid, esterified sugars, sugar polymers, monosaccharides, fructose, maltose, galactose, glucose, D-mannose, sorbose, disaccharides, lactose, sucrose, trehalose, cellobiose, polysaccharides, raffinose, melezitose, maltodextrins, dextrans, starches, citric acid, sodium chloride, potassium chloride, sodium sulfate, potassium nitrate, and sodium phosphate. In some embodiments, the antimicrobial agent comprises one or more of benzalkonium chloride, benzethonium chloride, benzyl alcohol, cetylpyridinium chloride, chlorobutanol, phenol, phenylethyl alcohol, phenylmercuric nitrate, and thimerosal. In some embodiments, the composition further comprises an antioxidant selected from ascorbyl palmitate, butylated hydroxyanisole, butylated hydroxytoluene, hypophosphorous acid, monothioglycerol, propyl gallate, sodium bisulfite, sodium formaldehyde sulfoxylate, and sodium metabisulfite. In some embodiments, the composition further comprises a surfactant selected from polysorbates, sorbitan esters, lecithin, phosphatidylcholines, phosphatidylethanolamines, fatty acids, fatty acid esters and cholesterol. In some embodiments, the composition further comprises a freezing agent selected from 3% to 12% dimethylsulfoxide (DMSO) and 1% to 5% human albumin. In some embodiments, the composition further comprises a preservative selected from one or more of methylparaben, propylparaben, sodium benzoate, benzalkonium chloride, antioxidants, chelating agents, parabens, chlorobutanol, phenol, and sorbic acid.

In some embodiments, the invention is a method of inhibiting the growth of a tumor in a patient comprising administering to a patient having the tumor the composition described herein. In some embodiments, the tumor is a hematological tumor or any other tumor expressing CD371. In some embodiments, the hematological tumor is selected from acute myeloblastic leukemia (AML) and myelodysplastic syndrome (MDS).

In some embodiments, the administering is selected from the group consisting of systemic delivery, parenteral delivery, intramuscular delivery, intravenous delivery, subcutaneous delivery, and intradermal delivery. In some embodiments, the administered composition further comprises a delivery-timing component that enables time-release, delayed release, or sustained release of the composition. In some embodiments, the delivery-timing component is selected from monostearate, gelatin, a semipermeable matrix, and a solid hydrophobic polymer. In some embodiments, the method further comprises administering a cytokine to the patient. In some embodiments, the cytokine is selected from IL-21, IL-2 and IL-15.

In some embodiments, the method further comprises a step of measuring expression of CD371 in the cells of the tumor prior to the administering step. In some embodiments, the method further comprises, prior to administering to the patient, applying to the immune cells a quality control measure comprising assessing one or more properties selected from presence of the CAR in the cellular genome, surface expression of the CAR, antigen-dependent lysis of antigen-expressing target cells, proliferation in the presence of antigen-expressing target cells, cytokine secretion in the presence of antigen-expressing target cells, cell exhaustion and the presence of a memory cell phenotype. In some embodiments, the presence of the CAR in the cellular genome is assessed by a method selected from nucleic acid hybridization, nucleic acid sequencing, polymerase chain reaction (PCR), quantitative PCR (qPCR), real-time PCR (rtPCR) and droplet digital PCR (ddPCR). In some embodiments, the surface expression of the CAR is assessed by flow cytometry, fluorescence-activated cell sorting (FACS), microfluidics-based screening, ELISA, or Western blot. In some embodiments, the surface expression of the CAR is assessed by flow cytometry with an anti-FAB2 antibody. In some embodiments, the immune cell population with the highest surface expression of the CAR is selected for administration to the patient. In some embodiments, the antigen-dependent lysis of antigen-harboring target cells is assessed by co-culturing the immune cells with CD371 (CLL-1) expressing target cells at an effector:target ratio between about 0.1 and about 10 and assessing target cell lysis. In some embodiments, the immune cell population with the highest rate of lysis of antigen-harboring target cells is selected for administration to the patient. In some embodiments, the antigen-dependent proliferation is assessed by co-culturing the immune cells with CD371(CLL-1)-expressing target cells and assessing the proliferation of the immune cells. In some embodiments, the immune cell population with the highest rate of proliferation in the presence of target cells is selected for administration to the patient. In some embodiments, the secretion of one or more cytokines selected from gamma-interferon (IFNγ), tumor necrosis factor alpha (TNFα), IL-2, IL-4, IL-6 is assessed. In some embodiments, the cytokine secretion is assessed by co-culturing the immune cells with CD371 (CLL-1)-expressing target cells and measuring the amount of cytokines in the co-culture supernatant. In some embodiments, the immune cell population with the highest cytokine secretion is selected for administration to the patient. In some embodiments, the cell exhaustion is assessed by measuring expression of one or more of PD-1, LAG-3, TIM-3, CTLA-4, and the BLIMP-I transcription factor, and the TOX transcription factor. In some embodiments, the immune cell population with the lowest expression is selected for administration to the patient. In some embodiments, the cell exhaustion is assessed by measuring the rate of glycolysis, or oxidative phosphorylation, or a ratio of glycolysis to oxidative phosphorylation over time. In some embodiments, the immune cell population with the lowest glycolysis, or the lowest ratio of glycolysis to oxidative phosphorylation is selected for administration to the patient. In some embodiments, the memory phenotype is assessed by detecting a combination of cell surface markers comprising CCR7, CD45RA, CD45RO, CD62L, and CD27.

In some embodiments, the invention is a method of selecting a patient for treatment with the composition described herein, the method comprising measuring expression of CD371 (CLL-1) in the cells of the tumor. In some embodiments, the measuring is selected from qualitative and quantitative. In some embodiments, the expression is measured by a method selected from immunohistochemistry, flow cytometry, enzyme-linked immunosorbent assay (ELISA), Northern blotting, fluorescent in-situ hybridization (FISH), quantitative reverse-transcription polymerase chain reaction (qRT-PCR), antigen densitometry, and super-resolution microscopy. In some embodiments, the method further comprises administering the treatment if CD371 (CLL-1) expression is detected and not administering the treatment if the CD371 (CLL-1) expression is not detected. In some embodiments, the method further comprises administering the treatment if CD371 (CLL-1) expression is high and not administering the treatment if the CD371 (CLL-1) expression is low. In some embodiments, the method comprises establishing a threshold of CD371 (CLL-1) expression equal to statistical value. In some embodiments, the method comprises administering the treatment if CD371 (CLL-1) expression is at or above the threshold and not administering the treatment if the CD371 (CLL-1) expression is below the threshold.

### BRIEF DESCRIPTION OF THE DRAWINGS

FIG. 1 is a diagram of anti-CD371 (CLL-1) chimeric antigen receptors (CARs) having different antigen recognition regions.

FIG. 2 shows in vitro lysis of tumor cells by engineered CAR-T cells having the CARs shown in FIG. 1.

FIG. 3 is a diagram of anti-CD371 (CLL-1) chimeric antigen receptors (CARs) having the B10H5L antigen recognition region.

FIG. 4 shows in vitro lysis of tumor cells by engineered CAR-T cells having the CARs shown in FIG. 3.

FIG. 5 shows antigen-dependent proliferation of engineered anti-CD371 (CLL-1) CAR-T cells in the presence of tumor cells.

FIG. 6 shows IFNγ secretion by engineered anti-CD371 (CLL-1) CAR-T cells in the presence of tumor cells.

FIG. 7 shows TNFα secretion by engineered anti-CD371 (CLL-1) CAR-T cells in the presence of tumor cells.

FIG. 8 shows an in vivo study design to assess anti-tumor activity of engineered anti-CD371 (CLL-1) CAR-T cells.

FIG. 9 shows survival of tumor-engrafted mice treated with engineered anti-CD371 (CLL-1) CAR-T cells.

FIG. 10 shows tumor burden (measured as bioluminescence) in tumor-engrafted mice treated with engineered anti-CD371 (CLL-1) CAR-T cells.

FIG. 11 shows tumor burden (measured as bioluminescence) in tumor-engrafted mice treated with engineered anti-CD371 (CLL-1) CAR-T cells.

FIG. 12 shows changes in body weight of tumor-engrafted mice treated with engineered anti-CD371 (CLL-1) CAR-T cells.

FIG. 13 shows tumor burden (measured as bioluminescence) in tumor-engrafted mice treated with engineered anti-CD371 (CLL-1) CAR-T cells.

FIG. 14 shows tumor burden (measured as total flux) in tumor-engrafted mice treated with engineered anti-CD371 (CLL-1) CAR-T cells.

FIG. 15 shows average tumor burden (measured as Area Under the Curve, AUC) in tumor-engrafted mice treated with engineered anti-CD371 (CLL-1) CAR-T cells.

FIGS. 16A and 16B show results of assessing in vitro cytotoxicity of anti-CLL-1 CAR-T cells with B2M-HLA-E fusion and PDCD1 inactivation.

FIGS. 17A and 17B show results of assessing in vitro antigen-dependent cytokine release by anti-CLL-1 CAR-T cells with B2M-HLA-E fusion and PDCDJ inactivation.

FIGS. 18A and 18B show results of assessing antigen-dependent in vitro proliferation of anti-CLL-1 CAR-T cells with B2M-HLA-E fusion and PDCD1 inactivation.

FIGS. 19A and 19B show results of assessing the effect of PDCD1 inactivation on in vitro cytotoxicity after serial challenge of anti-CLL-1 CAR-T cells armored with a B2M-HLA-E fusion.

FIG. 20 shows results of assessing the effect of armoring via B2M-HLA-E fusion on competitive survival of anti-CLL-1 CAR-T cells with PDCDJ inactivation.

FIG. 21 shows results of assessing the effect of PDCD1 inactivation on in vivo anti-tumor activity of anti-CLL-1 CAR-T cells armored with a B2M-HLA-E fusion.

FIG. 22 shows results of assessing in vivo anti-tumor activity of anti-CLL-1 CAR-T cells with PDCD1 inactivation and armoring via B2M-HLA-E fusion.

### DETAILED DESCRIPTION OF THE INVENTION

### Definitions

The following definitions are provided to aid in understanding of the disclosure. Unless defined in this section, technical and scientific terms used in this disclosure have the meaning commonly understood by a person of ordinary skill in the art. See, e.g., Sambrook et al., Molecular Cloning, A Laboratory Manual, 4^{th} Ed. Cold Spring Harbor Lab. Press (2012).

The term “activation” refers to the state of a T-cell that includes one or both of cell proliferation and cytokine secretion by the cell.

The term “antibody” refers to an immunoglobulin molecule which specifically binds to an antigen. The term also refers to antibody fragments including Fv, Fab and F(ab)₂, scFv and other forms described e.g., in Antibodies: A Laboratory Manual, 2^{nd} Ed. Greenfield, E., ed., Cold Spring Harbor Lab. Press, N.Y. (2013).

The term “co-stimulatory domain” refers to a part of a T-cell receptor which is a binding partner that specifically binds a co-stimulatory ligand, thereby mediating a co-stimulatory response of the T-cell, proliferation, and cytokine secretion. Examples of co-stimulatory ligands include CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, and HVEM. Examples of co-stimulatory domains include CD27, CD28, 4-1BB, OX40, CD30, CD40, PD-1, ICOS, LFA-1, CD2, CD7, LIGHT, NKG2C, and B7-H3.

The term “therapeutic benefit” refers to an effect that improves the condition of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the tumor, or prevention of metastasis, or prolonging overall survival (OS) or progression free survival (PFS) of a subject with cancer.

The terms “pharmaceutically acceptable” and “pharmacologically acceptable” refer to molecular entities and compositions that do not produce an adverse, allergic, or other deleterious reaction in a patient. For example, the pharmaceutically and pharmacologically acceptable preparations should meet the standards set forth by the FDA Office of Biological Standards.

The term “pharmaceutically acceptable carrier” and “excipient” refer to aqueous solvents (e.g., water, aqueous solutions of alcohols, saline solutions, sodium chloride, Ringer's solution, etc.), non-aqueous solvents (e.g., propylene glycol, polyethylene glycol, vegetable oil, and injectable organic esters), as well as dispersion media, coatings, surfactants, gels, antioxidants, preservatives (e.g., antibacterial or antifungal agents, anti-oxidants, chelating agents, and inert gases), isotonic agents, absorption delaying agents, stabilizers, binders, disintegration agents, lubricants, sweetening agents, flavoring agents, and dyes. The concentration and pH of the various components in a pharmaceutical composition are adjusted according to well-known parameters for each component.

The term “domain” refers to one region in a polypeptide which is folded into a particular structure independently of other regions.

The term “effector function” refers to a specialized function of a differentiated cell, such as a NK cell.

The term “adoptive cell” refers to a cell that can be genetically modified for use in a cell therapy treatment. Examples of adoptive cells include T-cells, macrophages, and natural killer (NK) cells.

The term “cell therapy” refers to the treatment of a disease or disorder that utilizes genetically modified cells. The term “adoptive cell therapy (ACT)” refers to a therapy that uses genetically modified adoptive cells. Examples of ACT include T-cell therapies, CAR-T cells therapies, natural killer (NK) cell therapies and CAR NK cell therapies.

The term “lymphocyte” refers to a leukocyte that is part of the vertebrate immune system. Lymphocytes include T-cells such as CD4⁺ and/or CD8⁺ cytotoxic T-cells, alpha/beta T-cells, gamma/delta T-cells, and regulatory T-cells. Lymphocytes also include natural killer (NK) cells, natural killer T (NKT) cells, cytokine induced killer (CIK) cells, and antigen presenting cells (APCs), such as dendritic cells. Lymphocytes also include tumor infiltrating lymphocytes (TILs).

The terms “effective amount” and “therapeutically effective amount” of a composition such as a cell therapy composition, refer to a sufficient amount of the composition to provide the desired response in the patient to whom the composition is administered.

The terms “peptide,” “polypeptide,” and “protein” are interchangeable and refer to polymers of amino acids, including natural and synthetic (unnatural) amino acids, as well as amino acids not found in naturally occurring proteins, e.g., peptidomimetics, and D optical isomers. A polypeptide may be branched or linear and be interrupted by non-amino acid residues. The terms also encompass amino acid polymers that have been modified through acetylation, disulfide bond formation, glycosylation, lipidation, phosphorylation, cross-linking, or conjugation (e.g., with a label). The polypeptide need not include the full-length amino acid sequence of the reference molecule but can include only so much of the reference molecule as necessary in order for the polypeptide to retain its desired activity. For example, polypeptides comprising full-length proteins, fragments thereof, polypeptides with amino acid deletions, additions, and substitutions are encompassed by the terms “protein” and “polypeptide,” as long as the desired activity is retained. For example, polypeptides with 95%, 90%, 80%, or less of sequence identity with the reference polypeptide are included as long the desired activity is retained by the polypeptides.

The terms “CRISPR” (clustered regularly interspaced short palindromic repeats), “Cas” (CRISPR-associated protein) “CRISPR-Cas” and “CRISPR system” refer to the genome editing tool derived from prokaryotic organisms and comprising a nucleic acid guide molecule and a sequence-specific nucleic acid-guided endonuclease capable of cleaving a target nucleic acid strand at a site complementary to a sequence in the nucleic acid guide.

The term “NATNA” (nucleic acid targeting nucleic acid) refers to a nucleic acid guide molecule of the CRISPR system. NATNA may be comprised two nucleic acid targeting polynucleotides (“dual guide”) including a CRISPR RNA (crRNA) and transactivating CRISPR RNA (tracrRNA). NATNA may be comprised a single nucleic acid targeting polynucleotide (“single guide”) comprising crRNA and tracrRNA connected by a fusion region (linker). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

Acute myeloid leukemia (AML) accounts for about ⅓ of annual cases of leukemia but is responsible for nearly ½ of leukemia-related deaths in the U.S. Aggressive chemotherapy remains the mainstay of AML treatment but is poorly tolerated by senior patients. Small molecule inhibitors and an anti-CD33 antibody-drug conjugate (ADC) have also been used against AML.

Cellular immunotherapy with genetically modified immune cells (e.g., chimeric antigen receptor T-cells or CAR-T cells) has been successful in hematological cancers (see e.g., U.S. Pat. No. 9,464,140). The engineered immune cells must target an antigen present on the surface of tumor cells but not present (or present at lower levels) on the surface of normal cells.

CD371 (CEC12A, DCAL-2, MICL or CLL-1) is a transmembrane glycoprotein expressed on monocytes, granulocytes, natural killer (NK) cells, and basophils. High levels of CD371 (CLL-1) expression have been reported in acute myeloid leukemia (AML) and myelodysplastic syndrome (MDS), as well as on leukemic stem cells (LSC), but not on granulocyte-macrophage progenitors (GMPs) making it an attractive target for treatment of AML (see WO2021050857 and references cited therein).

Disclosed herein are methods and compositions for treatment of hematological cancers including AML and myelodysplastic syndrome with CD371 (CLL-1)-targeting engineered immune cells.

In some embodiments, the invention comprises adoptive cells and the use of adoptive cells in cellular immunotherapy. Adoptive cells of the instant invention include lymphocytes, such as T-cells and CAR-T cells, natural killer (NK) cells, and CAR NK cells.

The cells of the instant invention are allogeneic cells, i.e., cells isolated from a donor individual, i.e., a healthy human donor of either gender.

In some embodiments, the cells are isolated from a healthy donor using standard techniques. For example, lymphocytes can be isolated from blood, or from lymphoid organs such as the thymus, bone marrow, lymph nodes, and mucosal-associated lymphoid tissues (MALT). Techniques for isolating lymphocytes from such tissues are well known in the art, see, e.g., Smith, J. W. (1997) Apheresis techniques and cellular immunomodulation, Ther. Apher. 1:203-206. In some embodiments, isolated lymphocytes are characterized in terms of specificity, frequency and function. In some embodiments, the isolated lymphocyte population is enriched for specific subsets of T-cells, such as CD4⁺, CD8⁺, CD25⁺, or CD62L⁺. See, e.g., Wang et al., Mol. Therapy —Oncolytics (2016) 3:16015. In some embodiments, after isolation, the lymphocytes are activated in order to promote proliferation and differentiation into specialized lymphocytes. For example, T-cells can be activated using soluble CD3/28 activators, or magnetic beads coated with anti-CD3/anti-CD28 monoclonal antibodies.

In some embodiments, a quality control measure or characterization step is applied to the isolated lymphocytes. In some embodiments, the quality control measure includes determining the percentage in the composition of CD4⁺, CD8⁺, CD25⁺, or CD62L+ cells, or cells expressing any combination of the above markers by flow cytometry.

The present invention comprises a method of treatment with allogeneic engineered immune cells. In some embodiments, the cells are genetically modified lymphocytes (including T-cells and NK cells). In some embodiments, the cells described herein are genetically modified to express a chimeric antigen receptor (CAR). In some embodiments, the cells are CAR-T cells. In some embodiments, the cells are CAR NK cells.

A typical chimeric antigen receptor (CAR) comprises an extracellular domain comprising an antigen binding region, a transmembrane domain and one or more intracellular activation (co-stimulatory) domains. In some embodiments, the CAR also comprises a hinge domain. In some embodiments, the CAR also comprises a leader peptide directing the CAR to the cell membrane.

The CAR disclosed herein comprises an extracellular domain comprising an antigen binding region targeting CD371 (also known as CEC12A, DCAL-2, MICL and CLL-1). In some embodiments, the antigen binding region is derived from an antibody. In some embodiments, the antigen binding region is derived from a monoclonal antibody. In some embodiments, the antigen binding region comprises a single-chain variable fragment (scFv). An scFv comprises a variable region of an antibody light chain (V_{L}) linked to a variable region of an antibody heavy chain (V_{H}). In some embodiments, the V_{L} is linked to the V_{H} via a peptide linker.

A peptide linker generally comprises from about 5 to about 40 amino acids. The linker can be a naturally occurring sequence or an engineered sequence. For example, in some embodiments, the linker is derived from a human protein, e.g., an immunoglobulin selected from IgG, IgA, I IgD, IgE, or IgM. In some embodiments, the linker comprises 5-40 amino acids from the CH1, CH2, or CH3 domain of an immunoglobulin heavy chain. In some embodiments, the linker is a glycine and serine rich linker having the sequence (GₓS_{y})ₙ. Additional linker examples and sequences are disclosed in the U.S. Pat. No. 5,525,491 Serine-rich peptide linkers, U.S. Pat. No. 5,482,858 Polypeptide linkers for production of biosynthetic proteins, and a publication WO2014087010 Improvedpolypeptides directed against IgE. In some embodiments, the peptide linker comprises GGGS (SEQ ID NO: 1). In some embodiments, the peptide linker consists of SEQ ID NO: 1. In some embodiments, the peptide linker comprises GGGGS (SEQ ID NO: 2). In some embodiments, the peptide linker consists of SEQ ID NO: 2.

In some embodiments, the antigen-binding region is a single-chain variable fragment (scFv). In some embodiments, the scFv comprises an antibody heavy chain (V_{H}) and an antibody light chain (V_{L}) connected by an amino acid linker comprising the sequence GGGS (SEQ ID NO: 1). In some embodiments, the scFv comprises an antibody heavy chain (V_{H}) and an antibody light chain (V_{L}) connected by an amino acid linker consisting of the sequence GGGS (SEQ ID NO: 1). In some embodiments, the linker comprises the sequence GGGGS (SEQ ID NO: 2). In some embodiments, the linker consists of the sequence GGGGS (SEQ ID NO: 2). In some embodiments, the linker comprises the sequence such as SEQ ID NO: 1 or SEQ ID NO: 2 repeated one or more times, e.g., between 1 and about 5 times. In some embodiments, the linker consists of the sequence (GGGS)n where n is a number between 1 and about 5 (SEQ ID NO: 43). In some embodiments, the linker consists of the sequence (GGGGS)ₙ where n is a number between 1 and about 5 (SEQ ID NO: 44).

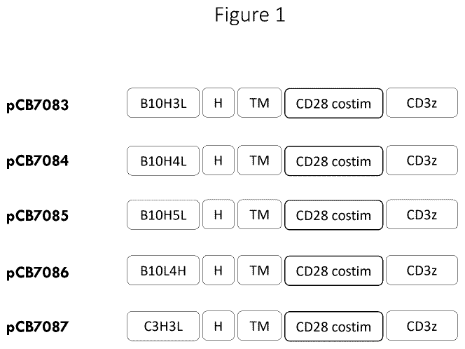

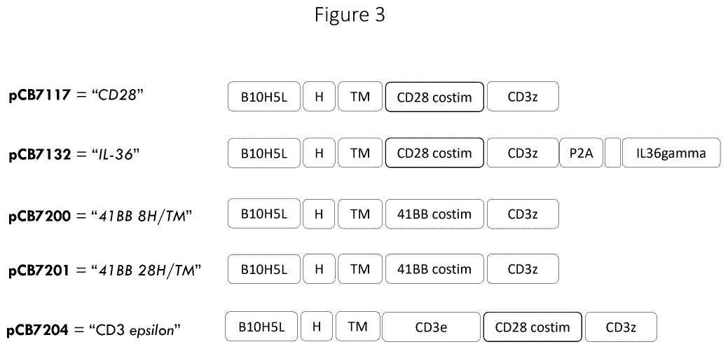

In some embodiments, the scFv structure in the N-C orientation is V_{H}-(linker)ₙ-V_{L}, where n is a number between 1 and about 5. In some embodiments, the scFv structure in the N-C orientation is V_{L}-(linker)ₙ-V_{H}, where n is a number between 1 and about 5. Examples of such CAR structures are shown in FIG. 1 and FIG. 3. For example, the scFv B10H3L (FIG. 1) comprises the V_{H} and V_{L} of the antibody B10 in the N-C orientation V_{H}-(linker)₃-V_{L}. The scFv B10L4H (FIG. 1) comprises the V_{H} and V_{L} of the antibody B10 in the N-C orientation V_{L}-(linker)₄-V_{H}.

In some embodiments, the CAR comprises an scFv described in the International Application Pub. No. WO2021050857 Anti-CD371 antibodies and uses thereof or the International Application Pub. No. WO2021050862 Antigen recognizing receptors targeting CD371 and uses thereof.

In some embodiments, the CAR comprises the scFv B10H5L described in WO2021050857. In some embodiments, the scFv comprises a sequence selected from SEQ ID NO: 3, 4 and 5, and 6. In some embodiments, the scFv consists of a sequence selected from SEQ ID NO: 3, 4 and 5, and 6.

In some embodiments, the antigen binding region comprises a heavy chain (V_{H}) comprising SEQ ID NO: 7. In some embodiments, the V_{H} comprises complementarity determining regions (CDR) 1, 2 and 3 comprising SEQ ID NOs.: 8, 9 and 10 respectively. In some embodiments, the antigen binding region comprises a light chain (V_{L}) comprising SEQ ID NO: 11. In some embodiments, the V_{L} comprises CDRs 1, 2 and 3 comprising SEQ ID NOs.: 12, 13 and 14 respectively.

In some embodiments, the antigen binding region comprises a heavy chain (V_{H}) consisting essentially of SEQ ID NO: 7. In some embodiments, the V_{H} comprises complementarity determining regions (CDR) 1, 2 and 3 consisting essentially of SEQ ID NOs.: 8, 9 and 10 respectively. In some embodiments, the antigen binding region comprises a light chain (V_{L}) consisting essentially of SEQ ID NO: 11. In some embodiments, the V_{L} comprises CDRs 1, 2 and 3 consisting essentially of SEQ ID NOs.: 12, 13 and 14 respectively.

In some embodiments, the CAR also comprises a hinge domain and the hinge domain is derived from CD8 or CD28 proteins. In some embodiments, the hinge domain comprises SEQ ID NO: 15. In some embodiments, the hinge domain consists essentially of SEQ ID NO: 15.

In some embodiments, the CAR comprises a signal peptide (a signal sequence) that enables trafficking of the CAR to the cell membrane. In some embodiments, the signal sequence comprises a CD28 signal sequence. In some embodiments, the signal sequence consists essentially of a CD28 signal sequence.

In some embodiments, the transmembrane domain of the CAR is derived from a membrane-bound or transmembrane protein. In some embodiments, the transmembrane domain is derived from the same protein as the co-stimulatory domains described below. For example, the transmembrane domain of the CAR may be the transmembrane domain of a T-cell receptor alpha-chain or beta-chain, a CD3-zeta chain, CD28, CD3-epsilon chain, CD45, CD4, CD5, CD8, CD9, CD16, CD22, CD33, CD37, CD64, CD80, CD86, CD134, CD137, ICOS, CD154, DNAM1, NKp44, NKp46, NKG2D, 2B4, or GITR. In some embodiments, the transmembrane domain is the CD8a transmembrane domain. In some embodiments, the transmembrane domain is the CD28 transmembrane domain. In some embodiments, the transmembrane domain comprises SEQ ID NO: 16. In some embodiments, the transmembrane domain consists essentially of SEQ ID NO: 16.

The cytoplasmic or intracellular signaling domain also referred to as the co-stimulatory domain of a CAR is responsible for activation of one or more effector functions of the immune cell expressing the CAR. In some embodiments, the co-stimulatory domain of the CAR comprises a part of or the entire sequence of the TCR zeta chain, CD3 zeta chain, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR4, TLR5, TLR6, MyD88, CD40 or a combination thereof. In some embodiments, the co-stimulatory domain of the CAR consists of a CD28 co-stimulatory domain. In some embodiments, the co-stimulatory domain of the CAR consists of a CD28 co-stimulatory domain. In some embodiments, the co-stimulatory domain of the CAR consists of a 4-1BB co-stimulatory domain. In some embodiments, the co-stimulatory domain of the CAR consists of a CD3epsilon co-stimulatory domain. In some embodiments, the co-stimulatory domain of the CAR is a combination of domains. In some embodiments, the co-stimulatory domain of the CAR consists of a CD3epsilon and a CD28 co-stimulatory domains. In some embodiments, the co-stimulatory domain of the CAR consists of a CD28 and a IL36gamma co-stimulatory domains. In some embodiments, the CAR comprises a P2A peptide cleavage site. In some embodiments, the cytoplasmic domain comprises a CD28 co0-stimulatory domain and a CD3 zeta chain. In some embodiments, the cytoplasmic domain comprises SEQ ID NO: 17. In some embodiments, the cytoplasmic domain consists essentially of SEQ ID NO: 17.

In some embodiments, the chimeric antigen receptor (CAR) comprises a sequence selected from SEQ ID NO.: 18, 19, 20, 21, 22, 23, 24, 25 and 26. In some embodiments, the CAR consists essentially of a sequence selected from SEQ ID NO: 18, 19, 20, 21, 22, 23, 24, 25 and 26. In some embodiments, the CAR is encoded by a sequence selected from SEQ ID NO: 27, 28, 29, 30,31,32,33,34 and 35.

In some embodiments, the CAR is fully human or is humanized to reduce immunogenicity in human patients. In some embodiments, the CAR sequence is optimized for codon usage in human cells.

The nucleic acid encoding the CAR (e.g., SEQ ID NO: 27-36) may be introduced into a cell as a genomic DNA sequence or a cDNA sequence. The cDNA sequence comprises an open reading frame for the translation of the protein (e.g., CAR) and in some embodiments, the cDNA further comprises untranslated elements that improve for example, the stability or the rate of translation of the CAR mRNA.

In some embodiments, the CAR coding sequence is inserted into the cellular genome into the endogenous T-cell receptor alpha chain (TRAC) gene. In some embodiments, the CAR is inserted into the TRAC locus on chromosome 14 approximately between nucleotides 22547529 and 22547552 (hg38). In some embodiments, the CAR is inserted into the TRAC locus on chromosome 14 approximately between nucleotides 22547538 and 22547539 (hg38). In some embodiments, the TRAC locus is targeted by a CRISPR-Cas endonuclease (e.g., Cas12a) and a guide polynucleotide having the targeting region of SEQ ID NO: 37 and the backbone of SEQ ID NO: 41.

In some embodiments, the cells used in the invention comprise the CAR and further comprise a genome modification resulting in armoring of the cells against an attack by the immune system of a recipient of the allogeneic immune cells (immune cells derived from a donor). In some embodiments, the armoring modification comprises protection from recognition by the cytotoxic T-cells of the host. Cytotoxic T-cells recognize MHC Class I antigens. An MHC Class I molecule is a cell surface molecule comprised of beta-2 microglobulin (B2M) associated with heavy chains of HLA-I proteins (selected from HLA-A, HLA-B, HLA-C, HLA-E, HLA-F and HILA-G). The B2M/HLA-I complex on the surface of the allogeneic cell is recognized by cytotoxic CD8+ T-cells and, if HLA-I is recognized as non-self, the allogeneic cell is killed by the T-cells. In some embodiments, the cells of the invention comprise an armoring genomic modification comprising a disruption of the B2M gene and therefore, disruption of the MHC Class I cell surface-bound complex. This disruption eliminates the MHC Class I antigen recognition that normally stimulates a cytotoxic T-cell attack.

In some embodiments, the armoring genome modification comprises disruption of recognition by the natural killer (NK) cells of the host. NK cells recognize cells without MHC-I protein as “missing self” and kill such cells. NK cells are inhibited by HLA-I proteins, including HLA-E, a minimally polymorphic HLA-I protein. In some embodiments, the cells of the invention comprise a first armoring genomic modification comprising a disruption of the B2M gene and therefore, disruption of the MHC Class I cell surface-bound complex, disruption of the MHC Class I antigen recognition that stimulates a cytotoxic T-cell attack, and further comprise a second armoring genomic modification comprising an insertion of an HLA-E gene fused to beta-2-microglobulin (B2M) gene, and therefore, expression of the HLA-E/B2M construct designed to cloak the cells from an attack by NK cells. See, e.g., Gornalusse et al., (2017) HLA-E-expressing pluripotent stem cells escape allogeneic responses and lysis by NK cells, Nat. Biotechnol. (2017) 35:765-772.

In some embodiments, the B2M-HLA-E insertion is in the B2M locus on chromosome 15 approximately between nucleotides 44715615 and 44715638 (hg38). In some embodiments, the B2M-HLA-E insertion is in the B2M locus on chromosome 15 approximately between nucleotides 44715624 and 44715625 (hg38).

Insertion of the B2M-HLA-E fusion into the B2M locus described herein may provide an in vivo survival advantage to T cells (including CAR-T cells) comprising the insertion compared to T cells or CAR-T cells not having the insertion or compared to T cells or CAR-T cells having a wild-type B2M locus. Without being bound by a particular theory, inventors attribute the survival advantage at least in part to reduced killing by the host's natural killer (NK) cells.

In some embodiments, survival advantage may be assessed by coculturing the T cells or CAR-T cells having the B2M-HLA-E fusion inserted into the B2M locus with natural killer (NK) cells. In some embodiments, a control experiment includes coculturing the T cells or CAR-T cells having wild-type B2M locus with natural killer (NK) cells. The number of live T cells or CAR-T cells in the coculture is assessed. In some embodiments, survival advantage due to insertion of the B2M-HLA-E fusion into the B2M locus is assessed by comparing the number of live T cells or CAR-T cells in the two cocultures. In some embodiments, survival advantage is assessed by coculturing the T cells or CAR-T cells having the B2M-HLA-E fusion inserted into the B2M locus as well as (in the same culture) T cells or CAR-T cells having wild-type B2M locus with natural killer (NK) cells. In some embodiments, survival advantage due to insertion of the B2M-HLA-E fusion into the B2M locus is assessed by comparing the number of live T cells or CAR-T cells with the fusion to the number of T cells or CAR-T cells with wild-type B2M locus in the same coculture.

In some embodiments, the armoring modification comprises transcriptionally silencing or disrupting one or more immune checkpoint genes. In some embodiments, the checkpoint gene is selected from PD-I (encoded by the PDCD1 gene), CTLA-4, LAG3, Tim3, BTLA, BY55, TIGIT, B7H5, LAIR1, SIGLEC10, and 2B4, see e.g., U.S. application publication US20150017136 Methods for engineering allogeneic and highly active T-cellfor immunotherapy.

Programmed cell death protein 1 (PD-1, encoded by the gene PDCD1), also known as CD279, is a cell surface receptor that plays an important role in downregulating the immune system, and promoting self-tolerance by suppressing T-cell inflammatory activity. PD-I binds to its cognate ligand, “programmed death-ligand 1,” also known as PD-L1, CD274, and B7 homolog 1 (B7-HI). PD-1 guards against autoimmunity through a dual mechanism of promoting programmed cell death (apoptosis) in antigen-specific T-cells in lymph nodes, while simultaneously reducing apoptosis in anti-inflammatory, suppressive T-cells (regulatory T-cells). Through these mechanisms, PD-1 binding of PD-L1 inhibits the immune system, thus preventing autoimmune disorders, but also prevents the immune system from killing cancer cells. Accordingly, mutating or knocking out production of PD-1 (e.g., by disrupting the PDCD1 gene) can be beneficial in T-cell therapies.

In some embodiments, the immune checkpoint gene is disrupted using an endonuclease that specifically cleaves nucleic acid strands within a target sequence of the gene to be disrupted. The strand cleavage by the sequence-specific endonuclease results in nucleic acid strand breaks that may be repaired by non-homologous end joining (NHEJ). NHEJ is an imperfect repair process that may result in direct re-ligation but more often, results in deletion, insertion, or substitution of one or more nucleotides in the target sequence. Such deletions, insertions, or substitutions of one or more nucleotides in the target sequence may result in missense or nonsense mutations in the protein coding sequence and eliminate production of any protein or cause production of a non-functional protein.

In some embodiments, the immune checkpoint gene is disrupted by contacting the cell with a sequence-specific endonuclease and triggering the NHEJ process within the cell resulting in elimination of protein expression of the immune checkpoint gene.

In some embodiments, the sequence-specific endonuclease is selected from a rare-cutting restriction enzyme, a TALEN, a Zinc-finger nuclease (ZFN) and a CRISPR endonuclease.

In some embodiments, the sequence-specific endonuclease is a CRISPR endonuclease selected from Cas9 and Casl2a. In some embodiments, the CRISPR endonuclease is part of a nucleoprotein complex comprising the CRISPR endonuclease and CRISPR guide RNA (nucleic acid targeting nucleic acid or NATNA). In some embodiments, the NATNA comprises one or more DNA nucleotides and is CRISPR hybrid R-DNA or chRDNA. In some embodiments, the NATNA is selected from the embodiments described in U.S. Pat. No. 9,650,617. In some embodiments, the NATNA is selected from the embodiments described in the International Application Pub. No. WO2022086846 DNA-containing polynucleotides and guides for CRSIPR Type V systems, and methods of making and using the same.

In some embodiments, the armoring modification comprises targeted cleavage and repair of the PDCD1 gene resulting in gene inactivation. In some embodiments, the PDCDJ gene is disrupted by cleavage of the PDCD1 locus in exon 2 of the PDCD1 gene. In some embodiments, the PDCD1 gene is disrupted by cleavage of the PDCD1 locus on chromosome 2 approximately between nucleotides 241852860 and 241852883 (hg38). In some embodiments, the PDCD1 locus is targeted by a CRISPR-Cas endonuclease (e.g., Cas12a) and a guide polynucleotide having the targeting region of SEQ ID NO: 39 and the backbone of SEQ ID NO: 41.

Inhibiting expression of PD-1 by disrupting the PDCD1 gene as described herein may result in increased antitumor activity of T cells (including CAR-T cells) with disrupted PDCD1 compared to T cells or CAR-T cells having a wild-type PDCD1. Without being bound by a particular theory, inventors attribute the increased antitumor activity at least in part to reduced inhibition by PD-1 ligand PD-L1 expressed by the tumor.

In some embodiments, increased antitumor activity may be assessed by coculturing T cells or CAR-T cells with disrupted PDCD1 locus with tumor cells known to express PD-L1. In some embodiments, a control experiment includes coculturing T cells or CAR-T cells having wild-type PDCD1 locus with tumor cells known to express PD-L1. After one or more time intervals, the number of live tumor cells or tumor cell lysis is assessed. In some embodiments, increased antitumor activity due to disruption of the PDCD1 locus is assessed by comparing the number of live tumor cells or tumor cell lysis in the two cocultures. In some embodiments, the coculture of T cells or CAR-T cells with tumor cells is challenged with additional tumor cells one or more times.

In some embodiments, the invention comprises a method of producing the anti-CD371 (CLL-1) chimeric antigen receptor (CAR). In some embodiments, the nucleic acid encoding the CAR is introduced into a target cell where expression of the CAR is desired. In some embodiments, the introduced nucleic acid is selected from an expression vector containing the CAR-encoding sequence, an mRNA encoding the CAR, and a delivery vector containing the CAR-encoding donor sequence to be inserted into the cellular genome. In some embodiments, the target cells are contacted with the nucleic acid encoding the CAR in vitro, in vivo or ex vivo.

In some embodiments, the vector used to deliver the CAR-encoding nucleic acid is a viral vector (e.g., a retroviral vector, adenoviral vector, adeno-associated viral vector, or lentiviral vector). Suitable vectors are non-replicating in the target cells. In some embodiments, the vector is selected from or designed based on SV40, EBV, HSV, or BPV. The vector incorporates the protein expression sequences. In some embodiments, the expression sequences are codon-optimized for expression in mammalian cells. In some embodiments, the vector also incorporates regulatory sequences including transcriptional activator binding sequences, transcriptional repressor binding sequences, enhancers, introns, and the like. In some embodiments, the viral vector supplies a constitutive or an inducible promoter. In some embodiments, the promoter is selected from EF1α, PGK1, MND, Ubc, CAG, CaMKIIa, and β-Actin promoter. In some embodiments, the promoter is selected from the SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the β-interferon promoter, the hsp70 promoter and EF-1α promoter. In some embodiments, the promoter is an MND promoter.

In some embodiments, the viral vector supplies a transcription terminator.

In some embodiments, the vector is a plasmid selected from a prokaryotic plasmid, a eukaryotic plasmid, and a shuttle plasmid.

In some embodiments, the CAR is expressed in a eukaryotic cell, such as a mammalian or human T-cell or NK cell (or their precursor) and the vector is a plasmid comprising a eukaryotic promoter active in the desired cell type, a secretion signal, a polyadenylation signal, and a stop codon, and, optionally, one or more regulatory elements such as enhancer elements.

In some embodiments, the expression vector comprises one or more selection marker. In some embodiments, the selection markers are antibiotic resistance genes or other negative selection markers. In some embodiments, the selection markers comprise proteins whose mRNA is transcribed together with the fusion protein mRNA and the polycistronic transcript is cleaved prior to translation.

In some embodiments, the expression vector comprises polyadenylation signals. In some embodiments, the polyadenylation sites are SV-40 polyadenylation signals.

In some embodiments, the coding sequence of the CAR is introduced into the cells via a viral vector, such as e.g., AAV vector (AAV6) or any other suitable viral vector capable of delivering an adequate payload. In some embodiments, to facilitate homologous recombination, the coding sequence is joined to homology arms located 5′ (upstream) and 3′ (downstream) of the insertion site in the desired insertion site in the genome. In some embodiments, the homology arms are about 500 bp long. See Eyquem J., et al. (2017) Targeting a CAR to the TRAC locus with CRISPR/Cas9 enhances tumor rejection, Nature, 543:113-117. In some embodiments, the sequence coding for the CAR together with the homology arms are cloned into a viral vector plasmid. The plasmid is used to package the sequences into a virus.

In some embodiment, the cells such as T-cells or NK cells or precursors thereof are contacted with a viral vector so that the genetic material delivered by the vector is integrated into the genome of the target cell and then expressed in the cell or on the cell surface. Transduced and transfected cells can be tested to confirm transgene expression using methods well known in the art such as fluorescence-activated cell sorting (FACS), microfluidics-based screening, ELISA, or Western blot. For example, the cells can be tested by staining or by flow cytometry with CAR-specific antibodies.

The present invention involves manipulating nucleic acids, including genomic DNA and plasmid DNA that were isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of reagent and kits are commercially available for extracting nucleic acids (DNA or RNA) from biological samples, including products from BD Biosciences (San Jose, Cal.), Clontech (TaKaRa Bio.); Epicentre Technologies (Madison, Wisc.); Gentra Systems, (Minneapolis, Minn.); Qiagen (Valencia, Cal.); Ambion (Austin, Tex.); BioRad Laboratories (Hercules, Cal.); KAPA Biosystems (Roche Sequencing Solutions, Pleasanton, Cal.) and more.

In some embodiments, the invention involves intermediate purification or separation steps for nucleic acids, e.g., to remove unused reactants from the DNA. The purification or separation may be performed by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.).

In some embodiments, exogenous protein-coding nucleic acid sequences (e.g., CAR-coding sequences or sequences coding for the immune-cloaking B2M-HLA fusion protein) are introduced into a cell such as a T-cell or a T-cell precursor, an NK cell or an NK cell precursor. In some embodiments, the “naked” nucleic acids are introduced into lymphocytes by electroporation as described e.g., in U.S. Pat. No. 6,410,319.

In some embodiments, the cell comprises the CRISPR system. In some embodiments, the CRISPR system comprises a nucleic acid-guided endonuclease and nucleic acid-targeting nucleic acid (NATNA) guides (e.g., a CRISPR guide RNAs selected from tracrRNA, crRNA or a single guide RNA incorporating the elements of the tracrRNA and crRNA in a single molecule). In some embodiments, the components of the CRISPR system are introduced into the cells (e.g., a T-cell or a T-cell precursor) in the form of nucleic acids.

In some embodiments, the components of the CRISPR system are introduced into the cells (e.g., a T-cell or a T-cell precursor) in the form of DNA coding for the nucleic acid-guided endonuclease and NATNA guides. In some embodiments, the gene coding for the nucleic acid-guided endonuclease (e.g., a CRISPR nuclease selected from Cas9 and Cas12a) is inserted into a plasmid capable of propagating in the target cell. In some embodiments, the gene coding for the NATNA guides is inserted into a plasmid capable of propagating in the target cell.

In some embodiments, the nucleic acid-guided endonuclease and NATNA guides are introduced into the target cells (e.g., a T-cell or a T-cell precursor) in the form of RNA, e.g., the mRNA coding for the nucleic acid-guided endonuclease along with the NATNA guides.

In some embodiments, the nucleic acid-guided endonuclease and the NATNA guides are introduced into the target cells (e.g., a T-cell or a T-cell precursor) as a preassembled nucleoprotein complex. In some embodiments, the nucleic acid-guided endonuclease and the NATNA guides are introduced into the target cells (e.g., T-cells or T-cell precursors) via any combination of different means, e.g., the endonuclease is introduced as the DNA via a plasmid containing the gene encoding the endonuclease while the guides are introduced in its final format as RNA (or RNA containing DNA nucleotides).

In some embodiments, the nucleic acids encoding the nucleic acid-guided endonuclease and NATNA guides are introduced into the cells via electroporation.

In some embodiments, the nucleic acids coding for the nucleic acid-guided endonuclease are introduced into cells in the form of mRNA as described e.g., in the U.S. Pat. No. 10,584,352 via electroporation or viral pseudo-transduction as described therein.

In some embodiments, one or more of the coding sequences described herein are introduced into the genome of the cell with the aid of a sequence-specific endonuclease. In some embodiments, the endonuclease is a nucleic acid-guided endonuclease encoded by the CRISPR locus. The CRISPR (Clustered Regularly Interspaced Short Palindromic Repeats) genomic locus is found many prokaryotic genomes and provides resistance to invasion of foreign nucleic acids. Structure, nomenclature and classification of CRISPR loci are reviewed in Makarova et al., Evolution and classification of the CRISPR-Cas systems. Nature Reviews Microbiology. 2011 June; 9(6): 467-477.

Briefly, a typical CRISPR locus includes a number of short repeats regularly interspaced with spacers. The CRISPR locus also includes coding sequences for CRISPR-associated (Cas) genes. A spacer-repeat sequence unit encodes a CRISPR RNA (crRNA). In vivo, a mature crRNAs are processed from a polycistronic transcript referred to as pre-crRNA or pre-crRNA array. The repeats in the pre-crRNA array are recognized by Cas-encoded proteins that bind to and cleave the repeats liberating mature crRNAs. CRISPR systems perform cleavage of a target nucleic acid wherein Cas proteins and crRNA form a CRISPR ribonucleoproteins (crRNP). The crRNA molecule guides the crRNP to the target nucleic acid (e.g., a foreign nucleic acid invading a bacterial cell) and the Cas nuclease proteins cleave the target nucleic acid.

Type I CRISPR systems include means for processing the pre-crRNA array that include a multi-protein complex called CASCADE (CRISPR-associated complex for antiviral defense) comprised of subunits CasA, B, C, D and E. The Cascade-crRNA complex recognizes the target nucleic acid through hybridization of the target nucleic acid with crRNA. The bound nucleoprotein complex recruits the Cas3 helicase/nuclease to facilitate cleavage of target nucleic acid.

Type II CRISPR systems include a trans-activating CRISPR RNA (tracrRNA). The tracrRNA hybridizes to a crRNA repeat in the pre-crRNA array and recruits endogenous RNaseIII to cleave the pre-crRNA array. The tracrRNA/crRNA complex can associate with a nuclease, e.g., Cas9. The crRNA-tracrRNA-Cas9 complex recognizes the target nucleic acid through hybridization of the target nucleic acid with crRNA. Hybridization of the crRNA to the target nucleic acid activates the Cas9 nuclease. for target nucleic acid cleavage.

Type III CRISPR systems include the RAMP superfamily of endoribonucleases (e.g., Cas6) that cleave the pre-crRNA array with the help of one or more CRISPR polymerase-like proteins.

Type VI CRISPR systems comprise a different set of Cas-like genes, including Csf1, Csf2, Csf3 and Csf4 which are distant homologues of Cas genes in Type I-III CRISPR systems.

Type V CRISPR systems are classified into several different subtypes, including, e.g., V-A, V-B, V-C, V-D, V-E, V-F, V-G, V-H, V-I, V-J, V-K and V-U. See, e.g., Makarova et al. (Nat. Rev. Microbiol., 2020, 18:67-83) and Pausch et al. (Science, 2020, 369(6501):333-337). The V-A subtype encodes the Casl2a protein (formerly known as Cpf1). Cas12a has a RuvC-like nuclease domain that is homologous to the respective domain of Cas9, but lacks the HNH nuclease domain that is present in Cas9 proteins. Type V systems can comprise a single crRNA sufficient for targeting of the Cas12 to a target site, or a crRNA-tracrRNA guide pair for targeting of the Cas12 to a target site.

CRISPR endonucleases require a nucleic acid targeting nucleic acid (NATNA) also known as guide RNAs. The endonuclease is capable of forming a ribonucleoprotein complex (RNP) with one or more guide RNAs. In some embodiments, the endonuclease is a Type II CRISPR endonuclease and NATNA comprises tracrRNA and crRNA.

In some embodiments, NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, NATNA is an engineered guide RNA comprising one or more DNA residues (CRISPR hybrid RNA-DNA or chRDNA). In some embodiments, NATNA is selected from the embodiments described in U.S. Pat. No. 9,650,617. Briefly, some chRDNA for use with a Type II CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of and a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, CRISPR Type V systems described in the International Application Pub. No. WO2022086846 (DNA-containing polynucleotides and guides for CRSIPR Type V systems, and methods of making and using the same) are used. In some embodiments, the CRISPR guide RNA (including the chRDNA) comprises a targeting region targeting a desired locus in the genome is located 5′ of the backbone. In some embodiments, Cas12a chRDNA sequences listed in Table 1 are used.

TABLE 1
CRISPR Type V chRDNAs

| | | |
|---|---|---|
| Gene | SEQ | |
| target | ID NO | Sequence |

CRISPR RNA-DNA (chRDNA) targeting region

| | | |
|---|---|---|
| TRAC | 37 | TrAArUrUrUCrUrACrUCrUTGrUrArGArUGr |
| | | ArGrUrCrUrCrUrCrAGrCrUrGrGrUrArCrA |
| | | C |
| B2M | 38 | TrAArUrUrUCrUrACrUCrUTGrUrArGArUAr |
| | | GrUrGrGrGrGrGrUrGArArUrUrCrArGrUrG |
| | | T |
| PDCD1 | 39 | rUrAArUrUrUrCrUrArCrUrCrUTGrUrArGr |
| | | ArUGrCrArCrGrArAGrCrUrCrUrCrCrGrAr |
| | | UrGrUrG |

CRISPR RNA-DNA (chRDNA) backbone

| | | |
|---|---|---|
| n/a | 41 | GrArGrUrCrUrCrUrCrAGrCrUrGrGrUrArC |
| | | rAC |

rN refers to a ribonucleotide and N refers to a
deoxyribonucleotide

In some embodiments, the endonuclease used to introduce one or more of the genetic modifications described herein (e.g., gene inactivation or insertion of the CAR-coding sequences, armoring sequences such as B2M-HLA-E protein fusions) into the genome of a cell is a restriction endonuclease, e.g., a Type II restriction endonuclease.

In some embodiments, the endonuclease used to introduce one or more of the genetic modifications described herein is a catalytically inactive CRISPR endonuclease (e.g., catalytically inactive Cas9 or Cas12a) conjugated to the cleavage domain of the restriction endonuclease Fok I. (see e.g., Guilinger, J. P., et al., (2014). Fusion of catalytically inactive Cas9 to FokI nuclease improves the specificity of genome modification, Nature biotechnology, 32(6), 577-582.

In some embodiments the endonuclease the endonuclease used to introduce one or more of the genetic modifications described herein is a zinc finger nuclease (ZFN), or a ZFN-Fok I fusion. In such embodiments, the target sequence is about 22-52 bases long and comprises a pair of ZFN recognition sequences, each 9-18 nucleotides long, separated by a spacer, which is 4-18 nucleotides long. (See e.g., Kim Y.G., et al., (1996). Hybrid restriction enzymes: zinc finger fusions to FokI cleavage domain, Proc Natl Acad Sci USA. 93(3): 1156-1160.

In some embodiments, the endonuclease the endonuclease used to introduce one or more of the genetic modifications described herein is a transcription activator-like effector nuclease (TALEN), or a TALEN-Fok I fusion. In such embodiments, the target sequence is about 48-85 nucleotides long and comprises a pair of TALEN recognition sequences, each 18-30 bases long, separated by a spacer, which is 12-25 bases long. (See e.g., Christian M. et al., (2010) Targeting DNA double-strand breaks with TAL effector nucleases, Genetics. 186 (2): 757-61.

In some embodiments, a quality control measure assessing one or more properties of the engineered anti-CD371 (CLL-1) CAR-T-cells is applied to the cells prior to administering the cells to a patient.

In some embodiments, the assessed property of the CAR-T cells is the presence of the CAR in the cellular genome. The presence of the CAR in the cellular genome may be assessed by a method selected from nucleic acid hybridization, nucleic acid sequencing and specific amplification including polymerase chain reaction (PCR), quantitative PCR (qPCR), real-time PCR (rtPCR) and droplet digital PCR (ddPCR). In some embodiments, the presence of the CAR in the cellular genome is assessed by ddPCR with amplification primers specific for one or both CAR insertion sites.

In some embodiments, the assessed property of the CAR-T cells is surface expression of the CAR. The surface expression of the CAR may be assessed by fluorescence-activated cell sorting (FACS), microfluidics-based screening, ELISA, or Western blot. In some embodiments, the surface expression of the CAR is assessed by flow cytometry with an anti-FAB2 antibody. In some embodiments, the CAR-T cell population with the highest surface expression of the CAR is selected for administration to a patient.

In some embodiments, the fraction of cells harboring the CAR in the genome or the fraction of cells expressing the CAR on the cell surface is used to determine the total number of cells constituting a therapeutically effective dose.

In some embodiments, the properties of the CAR-T cells are assessed in vitro and are selected from antigen-dependent lysis of antigen-expressing target cells (antigen-specific lysis); proliferation in the presence of antigen-expressing target cells (antigen-dependent proliferation); and cytokine secretion in the presence of antigen-expressing target cells, cell exhaustion and the presence of a memory cell phenotype.

In some embodiments, the in vitro assessment of CAR-T cells utilizes target cells or target cell lines. In some embodiments, the target cells are tumor cells selected from primary tumor cells and established tumor cell lines. In some embodiments, the tumor cells are known to express the specific antigen for the CAR-T cell, i.e., the tumor cells express CD371 (CLL-1) recognized by the anti-CD371 CAR-T cells. In some embodiments, the tumor cells are from tumor cell lines U937 or THP-1. In some embodiments, a control cell line, identical to the test cell line but lacking the specific antigen is used. In some embodiments, the control cell line harbors an inactivated gene coding for CD371 (a CD371 KO cell line).

In some embodiments, the assessed property is antigen-dependent lysis of antigen-harboring target cells. The antigen-dependent cell lysis may be assessed by co-culturing the population comprising engineered anti-CD371 (anti-CLL-1) CAR-T cells (effector cells or effectors) with a CD371 (CLL-1) expressing target cells (targets). The co-culture may be established at different effector:target ration (E:T ratios). In some embodiments, the E:T ratios are in the range of about 0.1 and about 10. In some embodiments, two or more E:T ratios in the selected range are evaluated. In some embodiments, cell lysis is detected by labeling target cells with cell permeant stable fluorescent dyes (e.g., CellTrace™ Violet (CTV), ThermoFisher Scientific, Carlsbad, Cal.). The fraction of live target cells was determined by incorporation of the viability dye by effector CAR-T cells. In some embodiments, a control experiment measures lysis of target cells lacking the antigen.

In some embodiments, the CAR-T cell population effecting the highest percentage of specific target cell lysis is selected for administration to a patient. In some embodiments, the CAR-T cell population effecting a high percentage of specific target cell lysis but having low non-specific target cell lysis is selected for administration to a patient.

In some embodiments, the assessed property is antigen-dependent proliferation of CAR-T cells. Proliferation may be assessed by co-culturing a population comprising engineered anti-CD371 (anti-CLL-1) CAR-T cells (effectors, E) with a CD371 (CLL-1)-expressing target cells (targets, T). In some embodiments, the co-culture is at E:T ratio of about 1. In some embodiments, cell proliferation is detected by labeling CAR-T cells with cell permeant stable fluorescent dyes (e.g., CellTrace™ Violet) and measuring dye dilution within the CAR-T cell population. In some embodiments, the CAR-T cell population exhibiting the highest rate of proliferation in the presence of target cells is selected for administration to a patient.

In some embodiments, the assessed property is cytokine secretion by CAR-T cells. In some embodiments, secretion of one or more cytokines is assessed. The one or more cytokines are selected from gamma-interferon (IFNγ), tumor necrosis factor alpha (TNFα), IL-2, IL-4, IL-6, and non-cytokine molecules Granzyme A, Granzyme B, and perforin. Cytokine secretion may be assessed by co-culturing a population comprising engineered anti-CD371 (anti-CLL-1) CAR-T cells (effectors, E) with a CD371 (CLL-1)-expressing target cells (targets, T). In some embodiments, the co-culture is at E:T ratio of about 1. In some embodiments, the cytokines in the co-culture supernatant can be detected or quantitatively detected by an antibody-based or antibody conjugate-based assay such as Western blotting or ELISA and similar secondary antibody-based methods with colorimetric or fluorescent detection methods.

In some embodiments, the assessed property of the CAR-T cells is T-cell exhaustion. T-cell exhaustion is characterized by expression of one or more of PD-1, LAG-3, TIM-3, CTLA-4, the BLIMP-I transcription factor, and the TOX transcription factor. The expression of the one or more of the PD-1, LAG-3, TIM-3, CTLA-4, BLIMP-1, and TOX may be assessed by assessing quantitatively or qualitatively, the presence of one or more of the above proteins or the mRNA encoding one or more of the above proteins. T-cell exhaustion is also characterized by decreased metabolic fitness which may be assessed by measuring the rate of glycolysis or oxidative phosphorylation (mitochondrial respiration) or a ratio of glycolysis to oxidative phosphorylation over time.

The presence and amount of mRNA in CAR-T cells may be assessed by a method selected from nucleic acid hybridization, nucleic acid sequencing and specific amplification including reverse transcription polymerase chain reaction (RT-PCR), quantitative RT-PCR (qRT-PCR), real-time RT-PCR (rtRT-PCR) and droplet digital RT-PCR (ddRT-PCR). In some embodiments, T-cell exhaustion is assessed by assessing the presence and optionally, the amount of the one or more of the PD-1, LAG-3, TIM-3, CTLA-4, BLIMP-1, and TOX mRNAs is assessed by ddPCR with amplification primers specific for the mRNA being assessed.

The presence and amount of the one or more of the PD-1, LAG-3, TIM-3, CTLA-4, BLIMP-1, and TOX proteins in CAR-T cells may be assessed by a method selected from flow cytometry inducing fluorescence-activated cell sorting (FACS), microfluidics-based screening, ELISA, or Western blot. In some embodiments, T-cell exhaustion is assessed by assessing the presence and optionally, the amount of the one or more of the PD-1, LAG-3, TIM-3, CTLA-4, BLIMP-I and TOX proteins by flow cytometry or FACS with an antibody or antibodies directed against said proteins.

The rate of glycolysis in T-cells may be assessed by measuring mitochondrial respiration and glycolysis in the cells. In some embodiments, T-cell exhaustion is assessed by measuring oxygen consumption rate (OCR) and extracellular acidification rate (ECAR) of the cells by measuring the concentration of dissolved oxygen and free protons in the extracellular medium. Commercial analyzers of OCR and ECAR are available (e.g., from Agilent Technologies, Santa Clara, Cal.).

In some embodiments, the T-cells with the lowest expression of exhaustion markers are selected for administration to a patient. In some embodiments, the T-cells with the lowest rate of glycolysis or the lowest ratio of glycolysis to mitochondrial respiration are selected for administration to a patient.

In some embodiments, the assessed property of the CAR-T cells is T-cell memory phenotype. The effector cell memory phenotype is characterized by the combination of cell surface markers comprising CCR7⁻ CD45RA⁻ CD45RO⁻ CD62L⁻ CD27⁻. In some embodiments, the T-cell memory phenotype is assessed by flow cytometry or FACS with antibodies directed against CCR7, CD45RA, CD45RO, CD62L, and CD27.

In some embodiments, the properties of the CAR-T-cells are assessed in vivo and are selected from affecting characteristics of experimental animals carrying target tumor cells. In some embodiments, the target cells are tumor cells known to express CD371 (CLL-1) and experimental animals are mice engrafted with the tumor cells prior to being administered a dose of the anti-CD371 (anti-CLL-1) CAR-T cells. In some embodiments, the experimental animals are NGS mice engrafted with the U937 tumor cells. In some embodiments, the assessment of CAR-T cells comprises monitoring body weight, overall survival, and tumor burden of the mice engrafted with the tumor cells and administered a dose of the anti-CD371 (anti-CLL-1) CAR-T cells.

In some embodiments, the animals are engrafted with a fluorescently labeled tumor cell lines and tumor burden is assessed by measuring in vivo fluorescence (other mouse measurements).

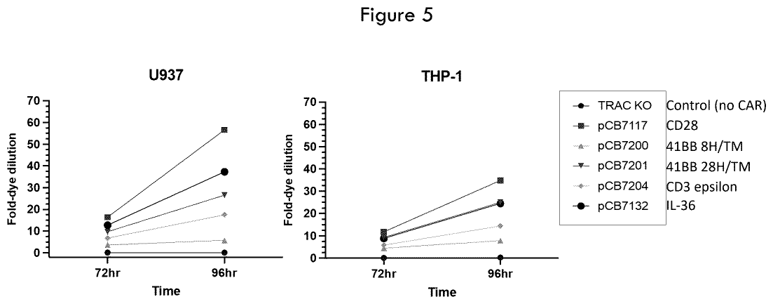

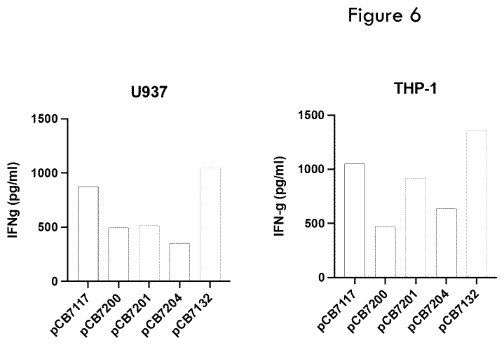

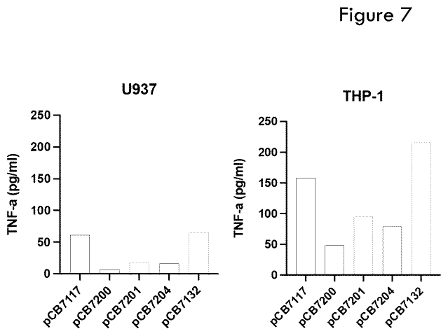

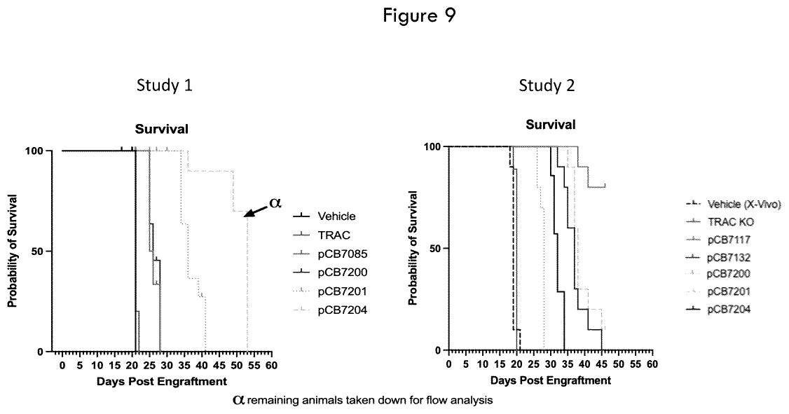

In some embodiments, a CAR-T cell clone is selected for inclusion into the therapeutic composition described herein. The inventors have discovered that surprisingly, the CAR-T cells engineered to express a CAR with the same anti-CD371 (anti-CLL-1) antigen binding region exhibit substantial variation in the properties assessed. Even more surprisingly, there can be poor correlation between the properties assessed in vitro and the anti-tumor activity assessed in vivo. (see FIGS. 5-7, 9-16). In some instances, a correctly inserted CAR was not expressed on the cell surface. For example, the clone pCB7203 (Example 2, Table 2) has the CAR inserted into the genome of the T-cells at a similar frequency as other clones. However, in the clone pCB7203, the CAR was expressed very poorly on the surface of the cell. Without being bound by a particular theory, the inventors attribute this failure to an unexpected phenomenon causing diminished or abrogated RNA expression, protein generation, or translocation of the protein to the cell surface. Similarly, clone pCB7204 had poor secretion of cytokines IFNγ and TNFα (FIGS. 6 and 7). However, surprisingly, the clone pCB7204 had one of the highest in vivo antitumor activities (FIGS. 9, 11A, 13B and 16).

In some embodiments, the invention comprises compositions including CAR-T cells exhibiting an anti-tumor property. In some embodiments, the invention comprises compositions including CAR-T cells assessed for having a satisfactory property or a satisfactory level of a parameter selected from one or more of: the presence of the CAR in the cellular genome, surface expression of the CAR, antigen-dependent cytotoxicity, antigen-dependent proliferation, cytokine secretion, expression of T-cell exhaustion markers, metabolic profile and expression of T-cell memory markers.

Once produced and (optionally) assessed for the desired properties, the engineered cells can be formulated into compositions for delivery to a human subject to be treated. The compositions include the engineered lymphocytes, and one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

In some embodiments, the composition further comprises an antimicrobial agent for preventing or deterring microbial growth. In some embodiments, the antimicrobial agent is selected from benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

In some embodiments, the composition further comprises an antioxidant added to prevent the deterioration of the lymphocytes. In some embodiments, the antioxidant is selected from ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some embodiments, the composition further comprises a surfactant. In some embodiments, the surfactant is selected from polysorbates, sorbitan esters, lipids, such as phospholipids (lecithin and other phosphatidylcholines), phosphatidylethanolamines, fatty acids and fatty esters; steroids, such as cholesterol.

In some embodiments, the composition further comprises a freezing agent such as 3% to 12% dimethylsulfoxide (DMSO) or 1% to 5% human albumin.

The number of CAR-T cells in the composition will vary depending on a number of factors but will optimally comprise a therapeutically effective dose per vial. A therapeutically effective dose can be determined experimentally by repeated administration of increasing amounts of the CAR-T cell-containing composition in order to determine which amount produces a clinically desired endpoint.

In some embodiments, where the subject is a human, the number of CAR-T-cells per dose is fewer than about 1×10⁸ of CAR-expressing cells. In some embodiments, the dose comprises between about 1×10⁵ cells/kg and 5×10⁶ cells/kg of body weight of the subject.

In some embodiments, the total number of cells in the dose is adjusted based on the percentage or CAR-expressing cells among all the cells in the cell composition. In some embodiments, the total number of cells administered is multiplied by 100/N where N is the percentage of CAR-expressing cells in the cell composition. The multiplication yields the total number of cells that must be administered to the patient in order to administer the desired number of CAR-expressing cells.

In some embodiments, the invention is a method of treating, preventing, or ameliorating a disease associated with expression of CD371 (CLL-1) comprising administering a population of immune cells (CAR-T cells or CAR NK cells) expressing the anti-CD371 (anti-CLL-1) CAR described herein.

In some embodiments, the population of immune cells administered to a patient has been assessed for having a satisfactory property or a satisfactory level of a parameter selected from one or more of: the presence of the CAR in the cellular genome, surface expression of the CAR, antigen-dependent cytotoxicity, antigen-dependent proliferation, cytokine secretion, expression of T-cell exhaustion markers, metabolic profile and expression of T-cell memory markers.

In some embodiments, the diseases or conditions that can be treated by the immune cells of the disclosure include various malignancies comprising hematological tumors selected from leukemia, AMIL and MDS.

In some embodiments, the invention is a method of inhibiting the growth of a tumor in a patient.

In some embodiments, the invention comprises a method of administering to a subject or patient a therapeutically effective number of immune cells expressing the anti-CD371 (anti-CLL-1) CAR described herein. In some embodiments, the immune cells are pre-activated and expanded prior to administration. In some embodiment, the administration of the immune cells according to the invention results in treating, preventing, or ameliorating the disease or condition in the subject or patient. In some embodiments, the disease or disorder is selected from cancers or tumors and infections that can be treated by administration of the immune cells that elicit an immune response.

A pharmaceutical composition comprising cells expressing the anti-CD371 (anti-CLL-1) CAR of the present disclosure can be delivered via various routes and delivery methods such as local or systemic delivery, including parenteral delivery, intramuscular, intravenous, subcutaneous, or intradermal delivery.

In some embodiments, the composition of the present invention is administered to a subject who has been preconditioned with an immunodepleting (e.g., lymphodepleting) therapy. In some embodiments, preconditioning is with lymphodepleting agents, including combinations of cyclosporine and fludarabine,

In some embodiments, the composition or formulation for administering to the patient is a pharmaceutical composition or formulation which permits the biological activity of an active ingredient and contains only non-toxic additional components such as pharmaceutically acceptable carriers. In some embodiments, pharmaceutically acceptable carriers include buffers, excipients, stabilizers, and preservatives.

In some embodiments, a preservative is used. In some embodiments, the preservative comprises one or more of methylparaben, propylparaben, sodium benzoate, benzalkonium chloride, antioxidants, chelating agents, parabens, chlorobutanol, phenol, and sorbic acid. In some embodiments, the preservative is present at about 0.0001% to about 2% by weight of the total composition.

In some embodiments, a carrier is used. In some embodiments, the carrier comprises a buffer, antioxidants including ascorbic acid and methionine; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA, sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the carrier comprises a buffer. In some embodiments, the buffer comprises citric acid, sodium citrate, phosphoric acid, potassium phosphate, and various other acids and salts. In some embodiments, the buffer is present at about 0.001% to about 4% by weight of the total composition.

In some embodiments, the pharmaceutical composition delivery systems such that the delivery of the composition occurs over time. In such embodiments the pharmaceutical composition comprises release-timing components. In some embodiments, the pharmaceutical composition comprises aluminum monostearate or gelatin. In some embodiments, the pharmaceutical composition comprises semipermeable matrices of solid hydrophobic polymers. In some embodiments, the matrices are in the form of films or microcapsules.

In some embodiments, the pharmaceutical composition comprises a sterile liquid such as an isotonic aqueous solution, suspension, emulsion, dispersions, or viscous composition, which may be buffered to a selected pH. In some embodiments, the pharmaceutical composition is a sterile injectable solution prepared by incorporating the cells in a solvent such as sterile water, physiological saline, or solutions or glucose, dextrose, or the like. In some embodiments, the pharmaceutical composition further comprises dispersing, or emulsifying agents, pH buffering agents, gelling or viscosity enhancing additives, preservatives, flavoring agents, colors, and the like, depending upon the route of administration and the preparation desired.

In some embodiments, the immune cells expressing the anti-CD371 (anti-CLL-1) CAR described herein are co-administered with cytokines. In some embodiments, the cytokines are selected from IL-2, IL-15 and IL21. In some embodiments, the cytokines are administered at a dose per kg of body weight of a human that is equivalent to 10 ng/mouse for IL-15, 100,000 units/mouse for IL-2, and 10 μg/mouse for IL-21.

In some embodiments, the invention comprises a diagnostic test to determine whether the patient is likely to benefit from treatment with anti-CD371 (anti-CLL-1) immune cells or not likely to benefit from the treatment. In some embodiments, the diagnostic test is administered prior to the treatment and is used to selecting or recommending the patient for the treatment.

In some embodiments, the invention comprises a method of treatment with the anti-CD371 (anti-CLL-1) immune cells described herein comprising a step of measuring expression of CD371 (CLL-1) in the cells of the tumor. In some embodiments, expression of CD371 (CLL-1) on the surface of the cells of the tumor is measured.

In some embodiments, the test is qualitative, i.e., detects the presence or absence of CD371 (CLL-1) expression (absence including any expression of CD371 (CLL-1) below the level of detection). In some embodiments, the test is quantitative, i.e., detects the level of CD371 (CLL-1) expression.

In some embodiments, the patient is selected for treatment with the anti-CD371 (anti-CLL-1) immune described herein if CD371 (CLL-1) expression is detected and the patient is advised against the treatment with the anti-CD371 (anti-CLL-1) immune cells described herein if CD371 (CLL-1) expression is not detected.

In some embodiments, the patient is selected for treatment with the anti-CD371 (anti-CLL-1) immune cells described herein if CD371 (CLL-1) expression is high and the patient is advised against the treatment with the anti-CD371 (anti-CLL-1) immune cells described herein if CD371 (CLL-1) expression is low.

In some embodiments, a threshold of CD371 (CLL-1) expression is established. In some embodiments, the threshold is equal to a top quantile in the population, such as the top half, top quartile, top 10% and so on. One of skill in the art is able to evaluate responses to the anti-CD371 (anti-CLL-1) therapy described herein in patients with various levels of CD371 (CLL-1) expression and determine which quantile is a threshold for CD371 (CLL-1) expression indicating the likelihood of a positive response to the anti-CD371 (anti-CLL-1) therapy described herein.

In some embodiments, the patient is selected for treatment with the anti-CD371 (anti-CLL-1) immune cell therapy described herein if CD371 (CLL-1) expression is at or above the threshold. In some embodiments, the patient is advised against the treatment with the anti-CD371 (anti-CLL-1) immune cells described herein if CD371 (CLL-1) expression is below the threshold.

Methods of quantitatively detecting protein expression in the cell, including on the cell surface are known in the art. In some embodiments, expression of CD371 (CLL-1) in the cells of the tumor is measured by a method detecting the CD371 (CLL-1) protein. Such methods include for example, immunohistochemistry, flow cytometry and enzyme-linked immunosorbent assay (ELISA). Anti-human CD371 (anti-human CLL-1) antibodies are available from multiple vendors including ThermoFisher Scientific, Miltenyi Biotech, BioLegend, BD Biosciences, Sony Biotechnology and more.

In some embodiments, expression of CD371 (CLL-1) is measured as presence of the CD371 (CLL-1) protein on the surface of the cells of the tumor. In some embodiments, the measurement is performed by a method selected from antigen densitometry and super-resolution microscopy.

In some embodiments, expression of CD371 (CLL-1) in the cells of the tumor is measured by a method detecting the mRNA encoding the CD371 (CLL-1) protein. Such methods include for example, Northern blotting, fluorescent in-situ hybridization (FISH), and quantitative reverse-transcription polymerase chain reaction (qRT-PCR).

### EXAMPLES

### Example 1. Designing and Engineering Anti-CD371 (CLL-1) CAR-T Cells

Diagrams of anti-CD371 (anti-CLL-1) CAR designs are shown in FIG. 1 and FIG. 3. Unless otherwise noted, each example below included a control cell line comprising no CAR but having a disrupted T-cell receptor alpha chain (TRAC) gene (“TRAC KO” control).

This Example describes the design and cloning of a DNA donor cassette into an AAV vector, production of AAV, delivery of Cas12a-chRDNA guide nucleoprotein complexes into primary cells, and transduction of primary cells with AAV for site-specific integration of a CAR expression cassette into primary cells.

A. In Silico Design of AAV Donor Cassettes and rAAV Production

The CAR designs are shown in FIG. 1 and FIG. 3. A mammalian promoter sequence was inserted upstream of the CAR polynucleotide. In order to site-specifically insert DNA donor polynucleotides into the host cell genome after site-specific cleavage, a target site was chosen in the endogenous IRAC locus on human chromosome 14 between nucleotides 22547538 and 22547539. Then, 500 bp long homology arms 5′ and 3′ of the cut site were identified. The 5′ and 3′ homology arms were appended to the end of the DNA donor polynucleotides, wherein the DNA donor polynucleotides were orientated in a reverse orientation (i.e., 3′ to 5′) relative to the homology arms.

The design for B2M and HLA class I histocompatibility antigen, alpha chain E (HLA-E), has been described. See, e.g., Gornalusse et al. Nature Biotechnology, 2017, 35(8):765-772 and the International Application Pub. No. WO2022086846 DNA-containing polynucleotides and guides for CRSIPR Type V systems, and methods of making and using the same. Briefly, the fusion nucleic acid construct encoded in the N-C orientation, an N-terminal B2M secretion signal, an HLA-G derived peptide sequence, a first linker sequence, the B2M sequence, a second linker sequence, an HLA-E sequence. The nucleic acid construct further contained an EF1a mammalian promoter sequence coding and a C-terminal BGH polyadenylation signal sequence.

In order to site-specifically insert DNA donor polynucleotide into the host cell genome after site specific cleavage, a target site was chosen in the endogenous B2M locus on human chromosome 15 between nucleotides 44715624 and 44715625. Then, 500 bp long homology arms 5′ and 3′ of the cut site were identified. The 5′ and 3′ homology arms were appended to the end of the DNA donor polynucleotides, wherein the DNA donor polynucleotides were oriented in a reverse orientation (i.e., 3′ to 5′) relative to the homology arms. The resulting DNA donor polynucleotide is presented in SEQ ID NO: 40, corresponding to SED ID NO: 414 in WO2022086846.

To target the PDCDJ gene, the exons were analyzed for the presence of a suitable PAM sequence, for example a 5′-TTTV-3′ PAM of the Type V Acidaminococcus spp., Cas12a (where ‘V’ is any nucleotide except thymine). A target site was chosen in the PDCD1 locus on human chromosome 2 between nucleotides 241852860 and 241852883. The 20 nucleotides 3′ of the PAM sequence were used for the generation of Cas12a guides, so that the 20 nucleotides 3′ of the PAM were included into a Cas12a crRNA guide.

Targeting regions of the guide polynucleotides (CRISPR hybrid RNA-DNA or chRDNA) are shown in Table 1 and are as follows: SEQ ID NO: 37 for the TRAC locus, SED ID NO: 38 for the B2Mlocus, and SEQ ID NO: 39 for the PDCDJ locus.

Oligonucleotide sequences coding for DNA donor polynucleotides were provided to a commercial manufacturer for synthesis into a suitable recombinant AAV (rAAV) plasmid. rAAV plasmids containing the nucleic acid constructs for the CAR designs in FIG. 1 and FIG. 3 and the B2M-HLA-E fusion were provided to a commercial manufacturer for packaging into two separate AAV6 viruses.

B. Primary T-Cell Transduction with rAAV

Primary activated T-cells were obtained from PBMCs as described in WO2022086846. Cas12a-chRDNA guide nucleoprotein complexes targeting the genes encoding TRAC and B2M were prepared also as described in WO2022086846.

Cell transfection and rAAV infection were also performed essentially as described in WO2022086846. Briefly, T-cells were transfected with gene-targeting Cas12a-chRDNA guide nucleoprotein complexes, and between 1 minute and 4 hours after nucleofection, cells were infected with the AAV6 virus packaged with donor sequences at an MOI of 1×10⁶. CAR donor sequences are listed as SEQ ID NOs: 28-36 and B2M-HLA-E donor sequence is listed as SEQ ID NO: 40. T-cells were cultured in ImmunoCult-XF complete medium (STEMCELL Technologies, Cambridge, Mass.) supplemented with IL-2 (100 units/mL) for 24 hours after the transductions. The next day, the transduced T-cells were transferred to 50 mL conical tubes and centrifuged at 300×g for approximately 7-10 minutes to pellet the cells. The supernatant was discarded, and the pellet was gently resuspended, and the T-cells pooled in an appropriate volume of ImmunoCult-XF complete medium supplemented with IL-2 (100 units/mL).

The enumerated T-cells were resuspended at 1×10⁶ cells/mL in ImmunoCult-XF complete medium supplemented with IL-2 (100 units/mL) and plated into as many T-175 suspension flasks as required (max volume per flask was 250 mL).

### Example 2. Detecting Anti-CD371 Anti-(CLL-1) CAR Expression in Engineered CAR-T Cells

In this example, the CAR-T cells engineered to express the anti-CD371 (anti-CLL-1) CAR as described in Example 1 were assessed for CAR expression by FACS with an anti-Fab2 antibody. The presence of the CAR in the cellular genome was also confirmed by PCR (droplet digital PCR, ddPCR) with primers specific for the left and right CAR intergradation sites in the TRAC gene. Results are shown in Table 2.

TABLE 2
Detecting CAR insertion by ddPCR and flow cytometry

| | |
|---|---|
| ddPCR | Flow cytometry |

| | Left % | Right % | TCR− | CAR+ |
|---|---|---|---|---|
| CAR T | Insertion | Insertion | % | % |
| TRAC KO | 0 | 0.1 | 95 | 0.5 |
| pCB7085 | 74 | 78 | 97 | 74 |
| pCB7200 | 75 | 77 | 98 | 95 |
| pCB7201 | 76 | 78 | 97 | 64 |
| pCB7203 | 78 | 78 | 97 | 5 |
| pCB7204 | 77 | 77 | 97 | 49 |

### Example 3. Specific Lysis of Tumor Cells by Anti-CD371 Anti-(CLL-1) CAR-T Cells

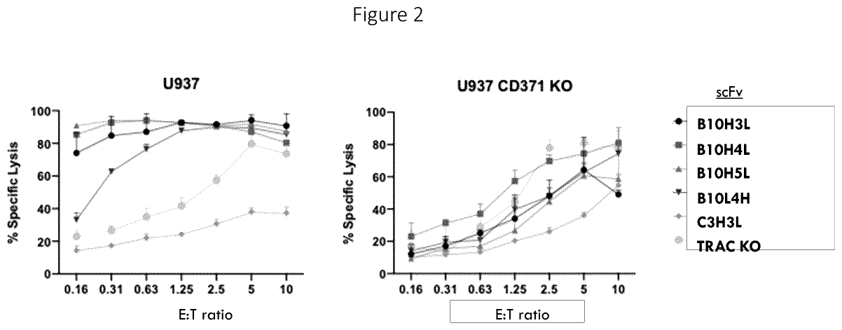

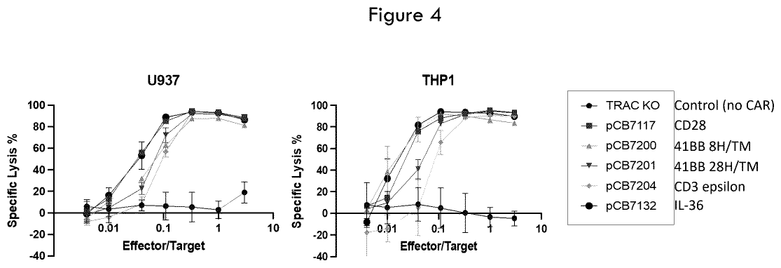

In this example, the CAR-T cells engineered to express the anti-CD371 (anti-CLL-1) CAR as described in Example 1 were cocultured with tumor cell lines U937 (human histiocytic lymphoma, ATCC CRL-1593.2) and THP-1 (human acute monocytic leukemia, ATCC TIB-202). Target cells were labelled with CellTrace™ Violet (CTV) (ThermoFisher Scientific, Carlsbad, Cal.) and co-cultured with effector cells at increasing E.T ratios for 48 hours. The fraction of live target cells was determined by T-cell incorporation of the viability dye. Results are shown in FIG. 2 and FIG. 4 for the CAR designs shown in FIG. 1 and FIG. 3 respectively.

### Example 4. Antigen-Dependent In Vitro Proliferation of Anti-CD371 (Anti-CLL-1) CAR-T Cells

In this example, the CAR-T cells engineered to express the anti-CD371 (anti-CLL-1) CAR as described in Example 1 were cocultured with tumor cell lines U937 and THP-1 (See Example 3). CAR-T cells were labeled with CellTrace™ Violet (CTV) and proliferation was measured by CTV dilution at 72 hr and 96 hr timepoints. Results are shown in FIG. 5.

### Example 5. In Vitro Antigen-Dependent Cytokine Release by Anti-CD371 (Anti-CLL-1) CAR-T Cells

In this example the CAR-T cells engineered to express the anti-CD371 (anti-CLL-1) CAR as described in Example 1 were cocultured with tumor cell lines U937 and THP-1 (See Example 3). Secretion of Interferon 7 (IFNγ) and Tumor Necrosis Factor α (TNFα) was measured by collecting supernatants from co-cultures at the 24 hr time point. Levels of IFN-g and TNF-α were quantified using a Luminex-based multiplex assay. Results are shown in FIG. 6 and FIG. 7.

### Example 6. In Vivo Antitumor Activity of Anti-CD371 (Anti-CLL-1) CAR-T Cells

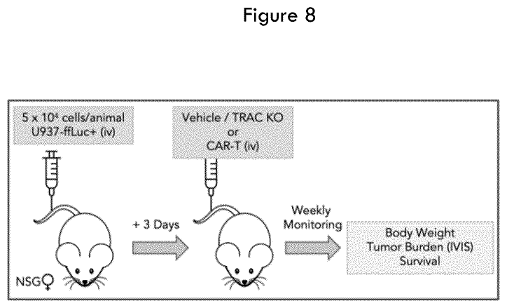

In this example, the CAR-T cells engineered to express the anti-CD371 (anti-CLL-1) CAR as described in Example 1 were injected into mice engrafted with U937 tumor cells (See Example 3). Experimental workflow is shown in FIG. 8. Three days prior to the CAR-T cell treatment, female NGS mice were injected intravenously with U937-ffLuc+ tumor cells at 5×10⁴ cells per animal. After 3 days of tumor engraftment, each animal was injected with 10⁷ CAR-expressing engineered anti-CD371 (anti-CLL-1) CAR-T cells of Example 1. The total number of cells injected was adjusted based on the percentage of CAR-expressing cell to reach the desired number of CAR-expressing cells in the injected dose. The negative controls included “TRAC-KO” (Example 1) and “Vehicle” consisting of 1:1 mixture of Plasma-lyte with 0.5% HSA and CryoStor™ CS10 medium. Experimental set up is shown in Table 3.

TABLE 3
Assessing anti-tumor activity of anti-
CD371 (anti-CLL-1) CAR-T-cells

| Group | | U937 | # | Injection | CAR |
|---|---|---|---|---|---|
| # | Treatment | cells | animals | volume | expression, % |
| 1 | Vehicle | 5 × 10⁴ | 10 | 200 uL, IV | N/A |
| 2 | TRAC KO | 5 × 10⁴ | 9 | 200 uL, IV | N/A |
| 3 | pCB7117 | 5 × 10⁴ | 10 | 200 uL, IV | 70 |
| 4 | pCB7132 | 5 × 10⁴ | 10 | 200 uL, IV | 63 |
| 5 | pCB7200 | 5 × 10⁴ | 10 | 200 uL, IV | 94 |
| 6 | pCB7201 | 5 × 10⁴ | 10 | 200 uL, IV | 67 |
| 7 | pCB7204 | 5 × 10⁴ | 7 | 200 uL, IV | 56 |

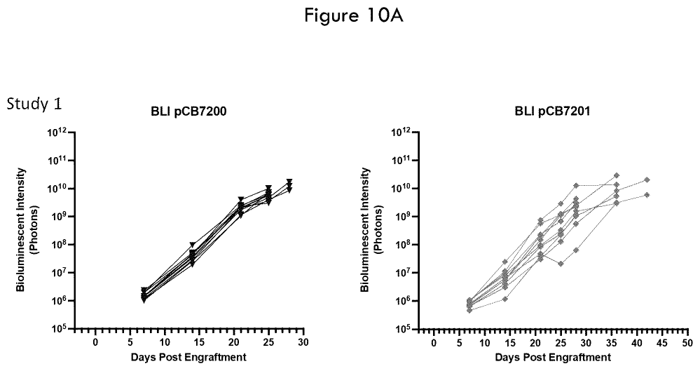

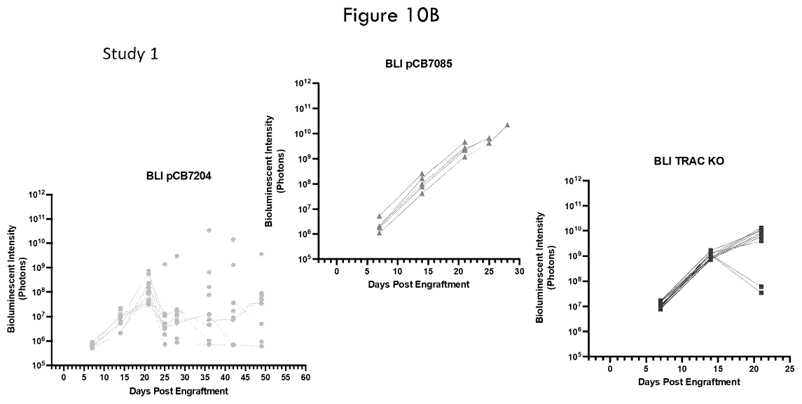

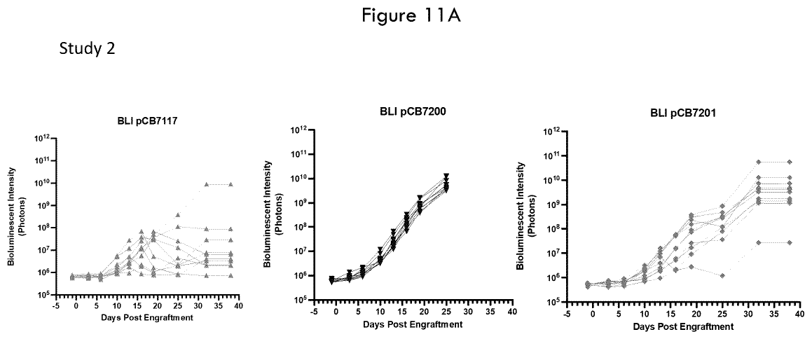

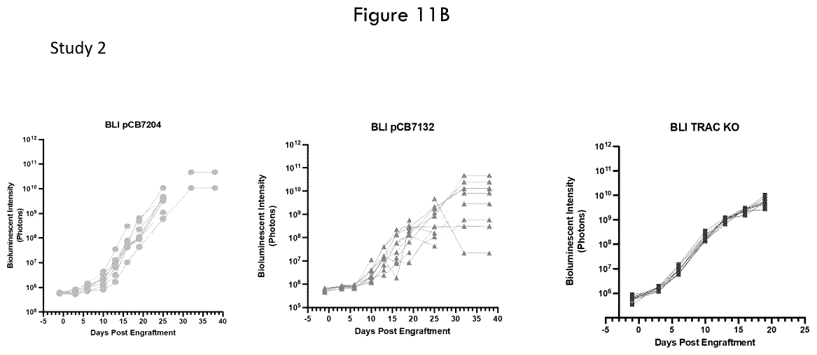

Different assessments of anti-tumor activity are shown in FIG. 9, FIG. 10, and FIG. 11.

FIG. 9 shows the probability of survival of the animals post-engraftment (Kaplan-Meier curves). The sign “a” marks a point at which the surviving animals (if any) were sacrificed for cytological analysis. Median survival is also shown in Table 4.

TABLE 4
Median survival post-engraftment

| Treatment | Median Survival, days |
|---|---|
| Vehicle | 21 |
| TRAC-KO | 21 |
| pCB7085 | 25.5 |
| pCB7200 | 26 |
| pCB7201 | 36 |
| pCB7204 | 53+ (remaining animals |
| | sacrificed at day 53) |

FIG. 10 and FIG. 11 show tumor burden in animals assessed post-engraftment assessed as bioluminescent intensity.

### Example 7. In Vivo Antitumor Activity of Anti-CD371 (Anti-CLL-1) CAR-T Cells

In this example the engineered anti-CD371 (anti-CLL-1) CAR-T cells were injected into mice engrafted with U937 tumor cells (See Example 3) as described in Example 6. Experimental set up is shown in Table 5. The animals were injected with one of the treatments listed in Table 5 on day 3 post-engraftment.

TABLE 4
Assessing anti-tumor activity of anti-
CD371 (anti-CLL-1) CAR-T-cells

| Group | | U937 | # | Injection | CAR |
|---|---|---|---|---|---|
| # | Treatment | cells | animals | volume | expression, % |
| 1 | Vehicle | 5 × 10⁴ | 10 | 200 uL, IV | N/A |
| 2 | TRAC KO | 5 × 10⁴ | 10 | 200 uL, IV | N/A |
| 3 | pCB7085 | 5 × 10⁴ | 5 | 200 uL, IV | 74 |
| 4 | pCB7201 | 5 × 10⁴ | 10 | 200 uL, IV | 95 |
| 5 | pCB7200 | 5 × 10⁴ | 10 | 200 uL, IV | 64 |
| 6 | pCB7204 | 5 × 10⁴ | 10 | 200 uL, IV | 49 |

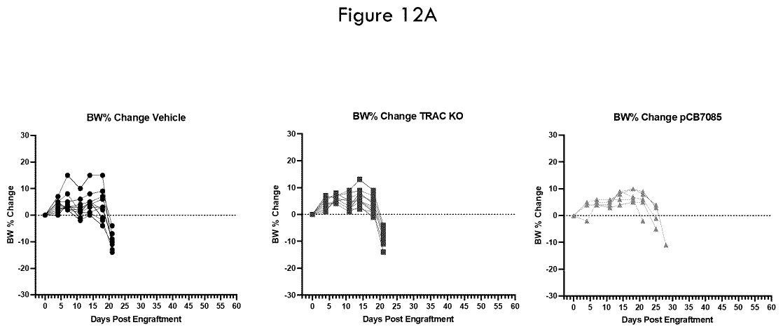

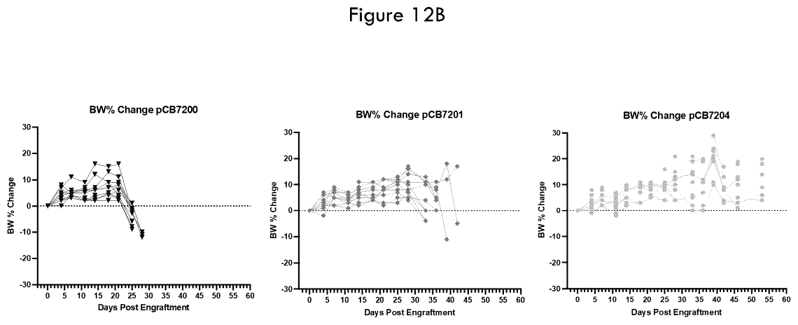

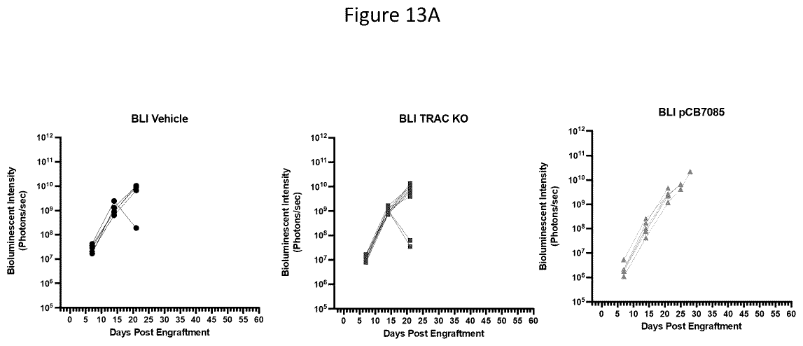

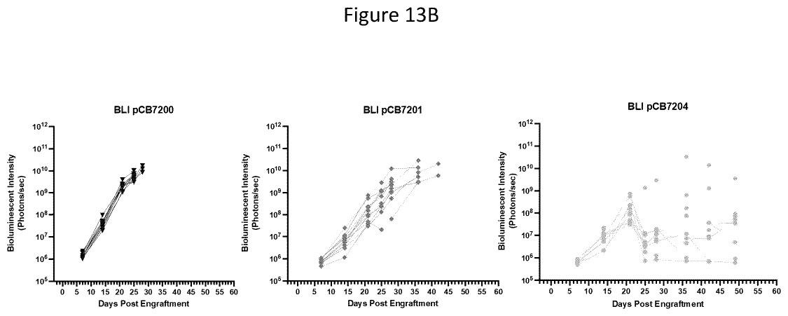

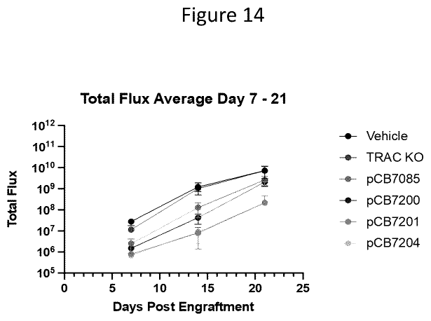

The mice were monitored for changes in body weight and tumor burden. Results are shown in FIG. 12, FIG. 13, and FIG. 14.

FIG. 12 shows body weight changes in the animals. The data is plotted as mean+/−standard deviation.

To assess tumor burden of U937-ffLuc+ in live mice, bioluminescence was measured weekly post-tumor engraftment starting with week 1 (day 7) and ending with week 7 (day 49). FIG. 13 shows bioluminescence intensity (photons per second) for each treatment group.

### Example 8. In Vivo Antitumor Activity of Anti-CD371 (Anti-CLL-1) CAR-T Cell Clone pCB7204

This example summarizes the data related to antitumor activity of anti-CD371 (anti-CLL-1) CAR-T cell clone pCB7204 and compares the antitumor activity of pCB7204 compared to other clones.

FIG. 14 shows a comparison of in vivo bioluminescence averages measured as total flux (imaged by IVIS® Spectrum in vivo imaging system as described in Example 7) for days 7-21 post engraftment.

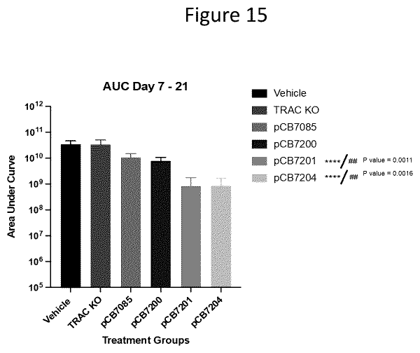

FIG. 15 shows a comparison of Area Under the Curve (AUC) calculated using the bioluminescence data from FIG. 9. The AUC analysis was performed to reduce the variability of bioluminescence data within treatment groups and to quantitatively understand the differences in bioluminescence between treatment groups. The p-values were calculated for pCB7201 (0.0011) and for pCB7204 (0.0016).

### Example 9. Specific Lysis of Tumor Cells (Cytotoxicity) by Anti-CLL-1 CAR-T Cells with B2M-HLA-E Fusion and PDCD1 Inactivation

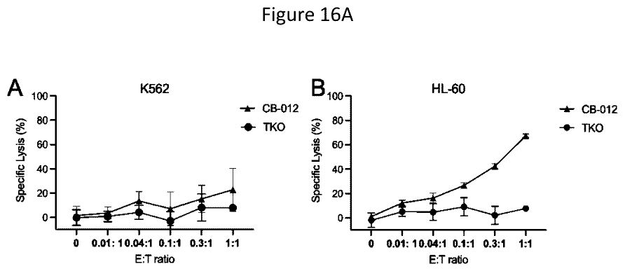

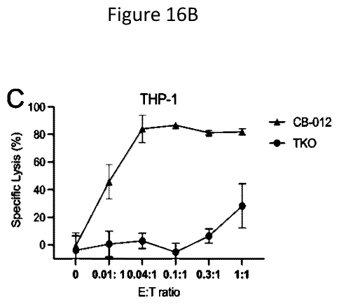

In this example, CAR-T cells were engineered to express the anti-CLL-1 CAR (CAR pCB7117, FIG. 3) and further engineered to express the B2M-HLA-E fusion and lack expression of PD-1 as described in Example 1. These cells are referred to as CB-012 (FIG. 16A and FIG. 16B). The cells were produced using large-scale manufacturing methods. The CAR-T cells were cocultured with CLL-1-expressing target tumor cell lines and target cell lysis was assessed. Control effector cells comprised disruption of TRAC, PDCD1 and B2M loci (triple knockout or TKO). The control cells had no CAR expression. The target cells were K562 cells, which do not express CLL-1, and CLL-1-expressing AML cell lines HL-60 and THP-1. Target cells were labelled with CellTrace™ Violet (CTV) (ThermoFisher Scientific, Carlsbad, Cal.) and co-cultured with effector cells at increasing E:T ratios (see FIG. 16A and FIG. 16B) for 48 hours. The fraction of live target cells was determined by T-cell incorporation of the viability dye which was released from lysed target cells. Specific lysis was calculated as 100%×(1−(count of live target cells in wells with effector cells/count of live target cells in target-only wells). Results are shown in FIG. 16A (K562 and HL-060) and FIG. 16B (THP-1).

### Example 10. In Vitro Antigen-Dependent Cytokine Release by Anti-CLL-1 CAR-T Cells with B2M-HLA-E Fusion and PDCD1 Inactivation

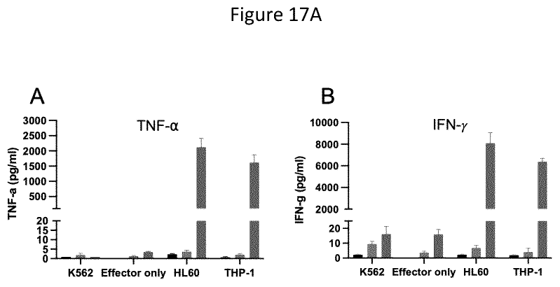

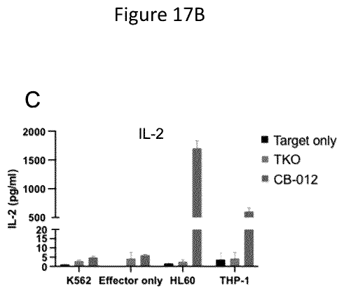

In this example, the CAR-T cells described in Example 9 (these cells are referred to as CB-012 in FIG. 17A and FIG. 17B) were cocultured with CLL-1-expressing target tumor cell lines and the presence of cytokines in the culture supernatant was assessed.

The cells were cocultured with tumor cell lines K562, HL60, and THP-1 (See Example 9) at the E:T ratio of 1:1. Triple knockout (TKO) effector cells (See Example 9) were used as a control. Supernatants from co-cultures were collected at the 24 hr time point and the presence of IL-2, Interferon γ (IFNγ) and Tumor Necrosis Factor α (TNFα) was measured using a Luminex-based multiplex assay quantitatively measuring the presence of each cytokine. Results are shown in FIG. 17A (TNFα and IFNγ) and FIG. 17B (IL-2).

### Example 11. Antigen-Dependent In Vitro Proliferation of Anti-CLL-1 CAR-T Cells with B2M-HLA-E Fusion and PDCD1 Inactivation

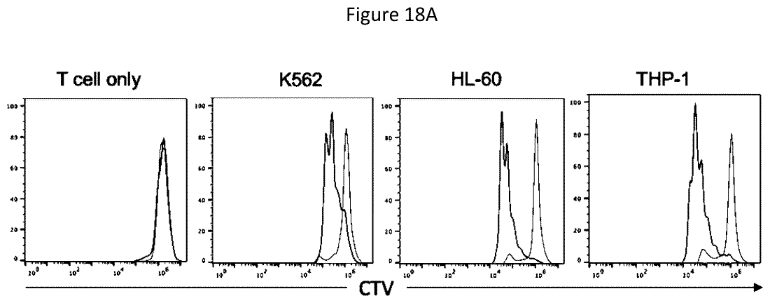

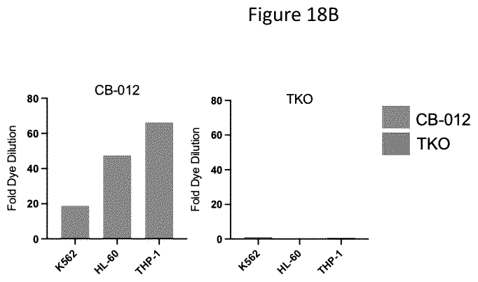

In this example, the CAR-T cells described in Example 9 (these cells are referred to as CB-012 in FIG. 18A) were cocultured with CLL-1-expressing target tumor cell lines and T cell proliferation was assessed.

Antigen-dependent proliferation of the CAR-T cells was evaluated in-vitro in response to co-culture with K562, HL-60 or THP-1 target cells (See Example 9) at a 1:1 effector to target ratio. Triple knockout (TKO) effector cells (See Example 9) were used as a control. T cells were labeled with CellTrace™ Violet (CTV) and proliferation was measured at 96 hours as a shift in CTV intensity from right to left on the X-axis due to dye dilution in progeny cells (FIG. 18A) and as dye dilution (FIG. 18B).

### Example 12. Effect of PDCD1 Inactivation on Antigen-Dependent In Vitro Cytotoxicity of Anti-CLL-1 CAR-T Cells with B2M-HLA-E Fusion

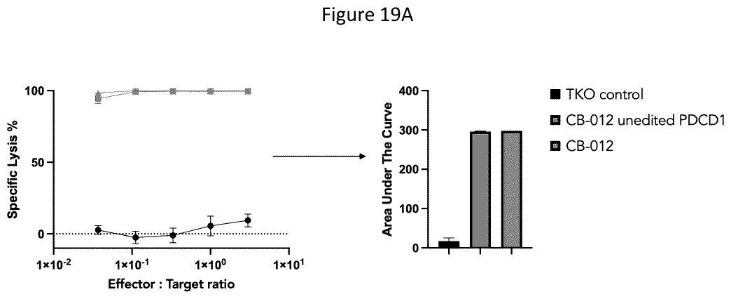

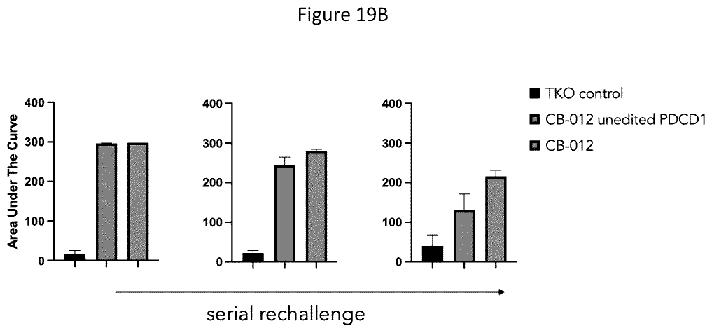

In this example, the CAR-T cells described in Example 9 were used (these cells are referred to as CB-012 in FIG. 19A and FIG. 19B). Control cells did not have the PDCD1 gene disrupted.

Cytotoxicity was assessed after repeat challenges of CAR-T cells with CLL-1-expressing target cell line U937. Target cells, which are engineered to express luciferase, were co-cultured with effector cells at increasing E:T ratios in the range of 1:100 to 10:1 (FIG. 19A) and live cells were assessed by a luminescence readout. Cytotoxicity specific lysis curves are transposed into plotting area under the curve AOC (upper right). Cytotoxicity was measured after 1, 4, and 6 rechallenges with CLL-1 expressing target cells. Results are shown in FIG. 19A and FIG. 19B.

### Example 13. Effect of Armoring Via B2M-HLA-E Fusion on Competitive Survival of Anti-CLL-1 CAR-T Cells with PDCD1 Inactivation

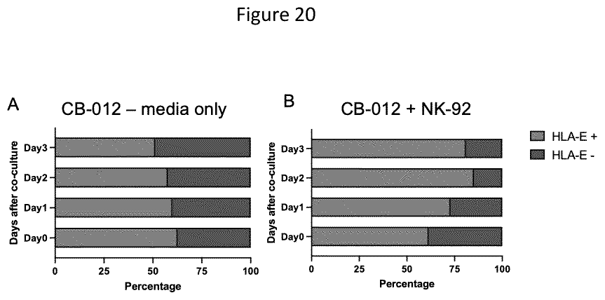

In this example, the CAR-T cells described in Example 9 were used (these cells are referred to as CB-012 in FIG. 20). Competitive survival of cells having the B2M-HLA-E fusion in the B2M locus (HLA-E⁺) within a mixed population with cells having the wild-type B2M locus (HLA-E⁻) was measured when cultured alone (A) or at a 1:1 ratio with cytotoxic NK-92 NK cells (B). The percentage of HLA-E+ cells in each culture was measured over 3 days by flow cytometry with anti-HLA-E antibody (BioLegend, San Diego, Cal.). Results are shown in FIG. 20.

### Example 14. Effect of PDCD1 Inactivation on In Vivo Anti-Tumor Activity of Anti-CLL-1 CAR-T Cells with B2M-HLA-E Fusion

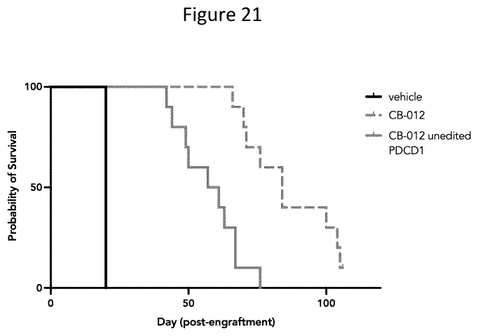

In this example, the CAR-T cells described in Example 9 were used (these cells are referred to as CB-012 in FIG. 21). Control cells did not have the PDCD1 gene inactivation.

Anti-CLL-1 CAR-T cells armored with B2M-HLA-E fusion and PDCD1 inactivation, anti-CLL-1 CAR-T cells armored with B2M-HLA-E fusion and having intact PDCD1 or vehicle were infused into NSG mice 3 days post-engraftment with U937 tumor cells overexpressing PD-L1. Probability of survival of the mice was plotted over time post-engraftment. Results are shown in FIG. 21 as Kaplan-Meier curves.

### Example 15. In Vivo Anti-Tumor Activity of Anti-CLL-1 CAR-T Cells with PDCD1 Inactivation and Armoring Via B2M-HLA-E Fusion

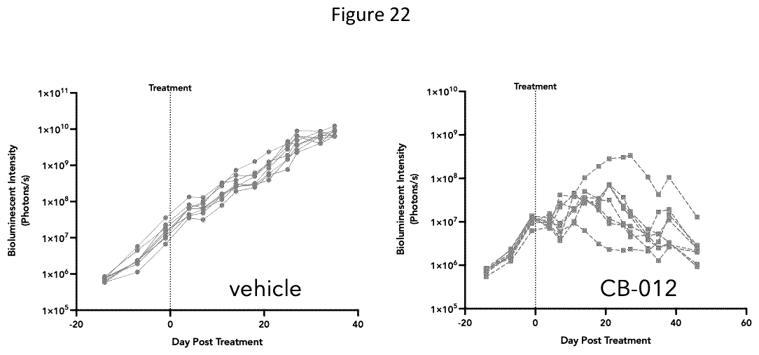

In this example, the CAR-T cells described in Example 9 were used (these cells are referred to as CB-012 in FIG. 22). The anti-CLL-1 CAR-T cells armored with B2M-HLA-E fusion and PDCD1 inactivation or vehicle was infused into NSG mice 21 days post-engraftment with HL-60 tumor cells. Tumor burden was assessed by bioluminescence intensity and plotted for individual animals over time. Results are shown in FIG. 22.

While the invention has been described in detail with reference to specific examples, it will be apparent to one skilled in the art that various modifications can be made within the scope of this invention. Thus, the scope of the invention should not be limited by the examples described herein, but by the claims presented below.

INFORMAL SEQUENCE LISTING

| SEQ ID | Name | Sequence |
|---|---|---|
| 1 | linker | GGGS |
| 2 | linker | GGGGS |
| 3 | B10H3L | EVQLLESGGGLVQPGGSLRLSCAASGFTFSDYQMSWVRQAPGKGLE |
| | scFv | WVSGIQGGGGSTYYADSVKGRFTISRDNSKNTLYLQMNSLRAEDTA |
| | | VYYCAREMWRGDYYSGMDVWGQGTTVTVSSGGGGSGGGGSGGGG |
| | | SDIVMTQSPDSLAVSLGERATINCKSSQSVLDSYNNENNLAWYQQKP |
| | | GQPPKLLIYWASTRESGVPDRFSGSGSGTDFTLTISSLQAEDVAVYYCQ |
| | | QYTSEPITFGQGTKVEIK |
| 4 | B10H4L | EVQLLESGGGLVQPGGSLRLSCAASGFTFSDYQMSWVRQAPGKGLE |
| | scFv | WVSGIQGGGGSTYYADSVKGRFTISRDNSKNTLYLQMNSLRAEDTA |
| | | VYYCAREMWRGDYYSGMDVWGQGTTVTVSSGGGGSGGGGSGGGS |
| | | GGGGSDIVMTQSPDSLAVSLGERATINCKSSQSVLDSYNNENNLAWY |
| | | QQKPGQPPKLLIYWASTRESGVPDRFSGSGSGTDFTLTISSLQAEDVAV |
| | | YYCQQYTSEPITFGQGTKVEIK |
| 5 | B10H5L | EVQLLESGGGLVQPGGSLRLSCAASGFTFSDYQMSWVRQAPGKGLE |
| | scFv | WVSGIQGGGGSTYYADSVKGRFTISRDNSKNTLYLQMNSLRAEDTA |
| | | VYYCAREMWRGDYYSGMDVWGQGTTVTVSSGGGGSGGGGSGGGG |
| | | SGGGSGGGGSDIVMTQSPDSLAVSLGERATINCKSSQSVLDSYNNENN |
| | | LAWYQQKPGQPPKLLIYWASTRESGVPDRFSGSGSGTDFTLTISSLQAE |
| | | DVAVYYCQQYTSEPITFGQGTKVEIK |
| 6 | B10L4H | DIVMTQSPDSLAVSLGERATINCKSSQSVLDSYNNENNLAWYQQKPG |
| | scFv | QPPKLLIYWASTRESGVPDRFSGSGSGTDFTLTISSLQAEDVAVYYCQ |
| | | QYTSEPITFGQGTKVEIKGGGGSGGGGSGGGSGGGGSEVQLLESGGG |
| | | LVQPGGSLRLSCAASGFTFSDYQMSWVRQAPGKGLEWVSGIQGGGG |
| | | STYYADSVKGRFTISRDNSKNTLYLQMNSLRAEDTAVYYCAREMWR |
| | | GDYYSGMDVWGQGTTVTVSS |
| 7 | V_{H} of | EVQLLESGGGLVQPGGSLRLSCAASGFTFSDYQMSWVRQAPGKGLE |
| | B10 | WVSGIQGGGGSTYYADSVKGRFTISRDNSKNTLYLQMNSLRAEDTA |
| | | VYYCAREMWRGDYYSGMDVWGQGTTVTVSS |
| 8 | CDR1 | GFTFSDYQ |
| | B10 V_{H} | |
| 9 | CDR2 | QGGGGST |
| | B10 V_{H} | |
| 10 | CDR3 | AREMWRGDYYSGMDV |
| | B10 V_{H} | |
| 11 | V_{L} of | DIVMTQSPDSLAVSLGERATINCKSSQSVLDSYNNENNLAWYQQKPG |
| | B10 | QPPKLLIYWASTRESGVPDRFSGSGSGTDFTLTISSLQAEDVAVYYCQ |
| | | QYTSEPITFGQGTKVEIK |
| 12 | CDR1 | QSVLDSYNNENN |
| | B10 V_{L} | |
| | CDR2 | WAS |
| | B10 V_{L} | |
| 14 | CDR3 | QQYTSEPIT |
| | B10 V_{L} | |
| 15 | Hinge | IEVMYPPPYLDNEKSNGTIIHVKGKHLCPSPLFPGPSKP |
| | CD8 or | |
| | CD28 | |
| 16 | TM CD8 | FWVLVVVGGVLACYSLLVTVAFIIFWV |
| | or CD28 | |
| 17 | Cytopl. | RSKRSRLLHSDYMNMTPRRPGPTRKHYQPYAPPRDFAAYRSRVKFSRS |
| | CD28 + | ADAPAYQQGQNQLYNELNLGRREEYDVLDKRRGRDPEMGGKPRRKN |
| | CD3 zeta | PQEGLYNELQKDKMAEAYSEIGMKGERRRGKGHDGLYQGLSTATKD |
| | | TYDALHMQALPPR |
| 18 | pCB7803 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSDIVMTQSPDSLAVSLGERATINCKSSQSVL |
| | | DSYNNENNLAWYQQKPGQPPKLLIYWASTRESGVPDRFSGSGSGTDF |
| | | TLTISSLQAEDVAVYYCQQYTSEPITFGQGTKVEIKAAAIEVMYPPPYL |
| | | DNEKSNGTIIHVKGKHLCPSPLFPGPSKPFWVLVVVGGVLACYSLLVT |
| | | VAFIIFWVRSKRSRLLHSDYMNMTPRRPGPTRKHYQPYAPPRDFAAYR |
| | | SRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQ |
| | | GLSTATKDTYDALHMQALPPR |
| 19 | pCB7804 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGSGGGSDIVMTQSPDSLAVSLGERATINCKSSQ |
| | | SVLDSYNNENNLAWYQQKPGQPPKLLIYWASTRESGVPDRFSGSGSG |
| | | TDFTLTISSLQAEDVAVYYCQQYTSEPITFGQGTKVEIKAAAIEVMYPP |
| | | PYLDNEKSNGTIIHVKGKHLCPSPLFPGPSKPFWVLVVVGGVLACYSL |
| | | LVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRD |
| | | PEMGGKPRRKNPQEGLYNELQKDKMAEAYSEIGMKGERRRGKGHDG |
| | | LYQGLSTATKDTYDALHMQALPPR |
| 20 | pCB7805 | MALPVTALLLPLALLLHAEVQLLESGGGLVQPGGSLRLSCAASGFTFS |
| | CAR | DYQMSWVRQAPGKGLEWVSGIQGGGGSTYYADSVKGRFTISRDNSK |
| | | NTLYLQMNSLRAEDTAVYYCAREMWRGDYYSGMDVWGQGTTVTVS |
| | | SGGGGSGGGGSGGGGSGGGSGGGGSDIVMTQSPDSLAVSLGERATIN |
| | | CKSSQSVLDSYNNENNLAWYQQKPGQPPKLLIYWASTRESGVPDRFS |
| | | GSGSGTDFTLTISSLQAEDVAVYYCQQYTSEPITFGQGTKVEIKEQKLIS |
| | | EEDLAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTR |
| | | KHYQPYAPPRDFAAYRSRVKFSRSADAPAYQQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPR |
| 21 | pCB7806 | MALPVTALLLPLALLLHAARPDIVMTQSPDSLAVSLGERATINCKSSQS |
| | CAR | VLDSYNNENNLAWYQQKPGQPPKLLIYWASTRESGVPDRFSGSGSGT |
| | | DFTLTISSLQAEDVAVYYCQQYTSEPITFGQGTKVEIKGGGGSGGGGS |
| | | GGGSGGGGSEVQLLESGGGLVQPGGSLRLSCAASGFTFSDYQMSWV |
| | | RQAPGKGLEWVSGIQGGGGSTYYADSVKGRFTISRDNSKNTLYLQM |
| | | NSLRAEDTAVYYCAREMWRGDYYSGMDVWGQGTTVTVSSAAAIEV |
| | | MYPPPYLDNEKSNGTIIHVKGKHLCPSPLFPGPSKPFWVLVVVGGVLA |
| | | CYSLLVTVAFIIFWVRSKRSRLLHSDYMNMTPRRPGPTRKHYQPYAPP |
| | | RDFAAYRSRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPR |
| 22 | pCB7117 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSGGGSGGGGSDIVMTQSPDSLAVSLGERATI |
| | | NCKSSQSVLDSYNNENNLAWYQQKPGQPPKLLIYWASTRESGVPDRF |
| | | SGSGSGTDFTLTISSLQAEDVAVYYCQQYTSEPITFGQGTKVEIKAAAIE |
| | | VMYPPPYLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAP |
| | | PRDFAAYRSRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPR |
| 23 | pCB7132 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSGGGSGGGGSDIVMTQSPDSLAVSLGERATI |
| | | NCKSSQSVLDSYNNENNLAWYQQKPGQPPKLLIYWASTRESGVPDRF |
| | | SGSGSGTDFTLTISSLQAEDVAVYYCQQYTSEPITFGQGTKVEIKAAAIE |
| | | VMYPPPYLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAP |
| | | PRDFAAYRSRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPRGSGATNFSLLKQAGDVEE |
| | | NPGPMYRMQLLSCIALSLALVTNSSMCKPITGTINDLNQQVWTLQGQN |
| | | LVAVPRSDSVTPVTVAVITCKYPEALEQGRGDPIYLGIQNPEMCLYCE |
| | | KVGEQPTLQLKEQKIMDLYGQPEPVKPFLFYRAKTGRTSTLESVAFPD |
| | | WFIASSKRDQPIILTSELGKSYNTAFELNIND |
| 24 | pCB7200 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSGGGSGGGGSDIVMTQSPDSLAVSLGERATI |
| | | NCKSSQSVLDSYNNENNLAWYQQKPGQPPKLLIYWASTRESGVPDRF |
| | | SGSGSGTDFTLTISSLQAEDVAVYYCQQYTSEPITFGQGTKVEIKAAAT |
| | | TTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVL |
| | | DKRRGRDPEMGGKPRRKNPQEGLYNELQKDKMAEAYSEIGMKGERR |
| | | RGKGHDGLYQGLSTATKDTYDALHMQALPPR |
| 25 | pCB7201 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSGGGSGGGGSDIVMTQSPDSLAVSLGERATI |
| | | NCKSSQSVLDSYNNENNLAWYQQKPGQPPKLLIYWASTRESGVPDRF |
| | | SGSGSGTDFTLTISSLQAEDVAVYYCQQYTSEPITFGQGTKVEIKAAAIE |
| | | VMYPPPYLDNEKSNGTIIHVKGKHLCPSPLFPGPSKPFWVLVVVGGVL |
| | | ACYSLLVTVAFIIFWVKRGRKKLLYIFKQPFMRPVQTTQEEDGCSCRFP |
| | | EEEEGGCELRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPR |
| 26 | pCB7204 | MALPVTALLLPLALLLHAARPEVQLLESGGGLVQPGGSLRLSCAASGF |
| | CAR | TFSDYQMSWVRQAPGKGLEWVSGIQGGGGSTYYADSVKGRFTISRDN |
| | | SKNTLYLQMNSLRAEDTAVYYCAREMWRGDYYSGMDVWGQGTTVT |
| | | VSSGGGGSGGGGSGGGGSGGGSGGGGSDIVMTQSPDSLAVSLGERATI |
| | | NCKSSQSVLDSYNNENNLAWYQQKPGQPPKLLIYWASTRESGVPDRF |
| | | SGSGSGTDFTLTISSLQAEDVAVYYCQQYTSEPITFGQGTKVEIKAAAIE |
| | | VMYPPPYLDNEKSNGTIIHVKGKHLCPSPLFPGPSKPFWVLVVVGGVL |
| | | ACYSLLVTVAFIIFWVKNRKAKAKPVTRGAGAGGRQRGQNKERPPPV |
| | | PNPDYEPIRKGQRDLYSGLNQRRIRSKRSRLLHSDYMNMTPRRPGPTR |
| | | KHYQPYAPPRDFAAYRSRVKFSRSADAPAYQQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPR |
| 27 | pCB7117 | ATGGCGTTGCCCGTGACCGCACTTCTGCTTCCATTGGCACTCCTGCT |
| | CAR | CCACGCGGCAAGACCGGAGGTGCAGCTGTTGGAGTCTGGGGGAGG |
| | | CTTGGTACAGCCTGGGGGGTCCCTGCGACTCTCCTGTGCAGCCTCT |
| | | GGATTCACCTTTAGCGACTATCAGATGAGCTGGGTCCGCCAGGCTC |
| | | CAGGGAAGGGGCTGGAGTGGGTGTCAGGCATTCAGGGTGGCGGTG |
| | | GTAGCACATATTACGCAGACTCCGTGAAGGGCCGGTTCACCATCTC |
| | | CCGTGACAATTCCAAGAACACGCTGTATCTGCAAATGAACAGCCTG |
| | | CGTGCCGAGGACACGGCTGTGTATTACTGTGCGAGAGAGATGTGGC |
| | | GTGGGGACTACTACTCCGGTATGGACGTCTGGGGCCAGGGGACCAC |
| | | GGTCACCGTCTCCTCAGGTGGTGGTGGTTCAGGTGGTGGTGGTTCT |
| | | GGAGGGGGCGGTTCTGGCGGCGGCTCCGGTGGTGGTGGATCCGAC |
| | | ATCGTGATGACCCAGTCTCCAGACTCCCTGGCTGTGTCTCTGGGCG |
| | | AGCGTGCCACCATCAACTGCAAGTCCAGCCAGAGTGTTTTAGACAG |
| | | CTATAACAATGAGAACAATTTAGCTTGGTATCAGCAGAAACCAGGA |
| | | CAGCCTCCTAAGCTGCTCATTTACTGGGCATCTACCCGGGAATCCG |
| | | GGGTCCCTGACCGATTCAGTGGCAGCGGGTCTGGGACAGATTTCAC |
| | | TCTCACCATCAGCAGCCTGCAGGCTGAAGATGTGGCAGTTTATTAC |
| | | TGTCAGCAATATACCAGCGAACCTATCACGTTCGGCCAAGGTACCA |
| | | AGGTGGAAATCAAAGCCGCTGCCATAGAAGTCATGTATCCTCCGCC |
| | | TTACCTGGATAACGAGAAGAGTAACGGAACTATCATACACGTGAA |
| | | GGGCAAACACCTCTGCCCCAGTCCCTTGTTTCCCGGCCCCAGCAAG |
| | | CCATTCTGGGTTTTGGTAGTGGTGGGTGGCGTCTTGGCTTGTTATTC |
| | | TCTCCTTGTCACAGTGGCATTTATTATCTTCTGGGTACGCTCCAAAC |
| | | GAAGCCGACTCCTGCACAGTGACTACATGAATATGACACCCCGGCG |
| | | GCCTGGTCCCACTAGGAAACATTACCAGCCATATGCTCCTCCCAGG |
| | | GACTTTGCCGCATATCGGAGTAGAGTGAAATTTAGCCGGTCTGCTG |
| | | ACGCTCCGGCCTATCAGCAAGGGCAAAACCAACTTTACAATGAGCT |
| | | TAACCTGGGGAGGCGAGAGGAATATGATGTATTGGATAAGCGCCG |
| | | AGGGAGGGACCCAGAGATGGGAGGAAAACCGAGGAGAAAAAACC |
| | | CGCAAGAGGGGCTTTATAATGAACTGCAGAAAGATAAGATGGCGG |
| | | AGGCTTACAGCGAGATCGGGATGAAGGGAGAGAGACGCAGAGGG |
| | | AAAGGCCACGACGGTCTCTACCAAGGCCTGAGTACGGCCACGAAA |
| | | GATACATACGATGCCCTCCATATGCAGGCCCTGCCACCGAGG |
| 28 | pCB7132 | ATGGCGTTGCCCGTGACCGCACTTCTGCTTCCATTGGCACTCCTGCT |
| | CAR | CCACGCGGCAAGACCGGAGGTGCAGCTGTTGGAGTCTGGGGGAGG |
| | | CTTGGTACAGCCTGGGGGGTCCCTGCGACTCTCCTGTGCAGCCTCT |
| | | GGATTCACCTTTAGCGACTATCAGATGAGCTGGGTCCGCCAGGCTC |
| | | CAGGGAAGGGGCTGGAGTGGGTGTCAGGCATTCAGGGTGGCGGTG |
| | | GTAGCACATATTACGCAGACTCCGTGAAGGGCCGGTTCACCATCTC |
| | | CCGTGACAATTCCAAGAACACGCTGTATCTGCAAATGAACAGCCTG |
| | | CGTGCCGAGGACACGGCTGTGTATTACTGTGCGAGAGAGATGTGGC |
| | | GTGGGGACTACTACTCCGGTATGGACGTCTGGGGCCAGGGGACCAC |
| | | GGTCACCGTCTCCTCAGGTGGTGGTGGTTCAGGTGGTGGTGGTTCT |
| | | GGAGGGGGCGGTTCTGGCGGCGGCTCCGGTGGTGGTGGATCCGAC |
| | | ATCGTGATGACCCAGTCTCCAGACTCCCTGGCTGTGTCTCTGGGCG |
| | | AGCGTGCCACCATCAACTGCAAGTCCAGCCAGAGTGTTTTAGACAG |
| | | CTATAACAATGAGAACAATTTAGCTTGGTATCAGCAGAAACCAGGA |
| | | CAGCCTCCTAAGCTGCTCATTTACTGGGCATCTACCCGGGAATCCG |
| | | GGGTCCCTGACCGATTCAGTGGCAGCGGGTCTGGGACAGATTTCAC |
| | | TCTCACCATCAGCAGCCTGCAGGCTGAAGATGTGGCAGTTTATTAC |
| | | TGTCAGCAATATACCAGCGAACCTATCACGTTCGGCCAAGGTACCA |
| | | AGGTGGAAATCAAAGCCGCTGCCATAGAAGTCATGTATCCTCCGCC |
| | | TTACCTGGATAACGAGAAGAGTAACGGAACTATCATACACGTGAA |
| | | GGGCAAACACCTCTGCCCCAGTCCCTTGTTTCCCGGCCCCAGCAAG |
| | | CCATTCTGGGTTTTGGTAGTGGTGGGTGGCGTCTTGGCTTGTTATTC |
| | | TCTCCTTGTCACAGTGGCATTTATTATCTTCTGGGTACGCTCCAAAC |
| | | GAAGCCGACTCCTGCACAGTGACTACATGAATATGACACCCCGGCG |
| | | GCCTGGTCCCACTAGGAAACATTACCAGCCATATGCTCCTCCCAGG |
| | | GACTTTGCCGCATATCGGAGTAGAGTGAAATTTAGCCGGTCTGCTG |
| | | ACGCTCCGGCCTATCAGCAAGGGCAAAACCAACTTTACAATGAGCT |
| | | TAACCTGGGGAGGCGAGAGGAATATGATGTATTGGATAAGCGCCG |
| | | AGGGAGGGACCCAGAGATGGGAGGAAAACCGAGGAGAAAAAACC |
| | | CGCAAGAGGGGCTTTATAATGAACTGCAGAAAGATAAGATGGCGG |
| | | AGGCTTACAGCGAGATCGGGATGAAGGGAGAGAGACGCAGAGGG |
| | | AAAGGCCACGACGGTCTCTACCAAGGCCTGAGTACGGCCACGAAA |
| | | GATACATACGATGCCCTCCATATGCAGGCCCTGCCACCGAGGGGTT |
| | | CTGGAGCAACCAACTTCTCCTTGCTGAAACAGGCCGGTGACGTGGA |
| | | GGAGAATCCCGGCCCTATGTACCGCATGCAACTTCTGTCTTGTATTG |
| | | CGCTTTCTCTCGCACTCGTAACCAATTCCAGCATGTGTAAGCCGATA |
| | | ACCGGGACTATAAACGATTTGAATCAGCAGGTATGGACCTTGCAGG |
| | | GACAGAATCTGGTAGCTGTACCACGGAGCGACAGCGTCACCCCCGT |
| | | GACTGTGGCCGTCATAACATGCAAATACCCGGAAGCGCTCGAACA |
| | | GGGCAGGGGTGATCCGATATACCTGGGAATCCAAAACCCGGAGAT |
| | | GTGCCTTTACTGCGAGAAGGTGGGCGAACAACCTACGCTTCAATTG |
| | | AAAGAGCAAAAGATAATGGACCTGTACGGCCAACCCGAACCCGTA |
| | | AAGCCCTTCCTCTTTTACCGCGCCAAGACTGGTAGAACAAGTACTC |
| | | TGGAGAGCGTTGCTTTTCCTGATTGGTTTATAGCAAGCTCCAAAAG |
| | | GGACCAGCCGATTATCCTCACAAGCGAACTCGGAAAGTCCTATAAT |
| | | ACCGCTTTTGAGTTGAATATCAATGAC |
| 29 | pCB7200 | ATGGCGTTGCCCGTGACCGCACTTCTGCTTCCATTGGCACTCCTGCT |
| | CAR | CCACGCGGCAAGACCGGAGGTGCAGCTGTTGGAGTCTGGGGGAGG |
| | | CTTGGTACAGCCTGGGGGGTCCCTGCGACTCTCCTGTGCAGCCTCT |
| | | GGATTCACCTTTAGCGACTATCAGATGAGCTGGGTCCGCCAGGCTC |
| | | CAGGGAAGGGGCTGGAGTGGGTGTCAGGCATTCAGGGTGGCGGTG |
| | | GTAGCACATATTACGCAGACTCCGTGAAGGGCCGGTTCACCATCTC |
| | | CCGTGACAATTCCAAGAACACGCTGTATCTGCAAATGAACAGCCTG |
| | | CGTGCCGAGGACACGGCTGTGTATTACTGTGCGAGAGAGATGTGGC |
| | | GTGGGGACTACTACTCCGGTATGGACGTCTGGGGCCAGGGGACCAC |
| | | GGTCACCGTCTCCTCAGGTGGTGGTGGTTCAGGTGGTGGTGGTTCT |
| | | GGAGGGGGCGGTTCTGGCGGCGGCTCCGGTGGTGGTGGATCCGAC |
| | | ATCGTGATGACCCAGTCTCCAGACTCCCTGGCTGTGTCTCTGGGCG |
| | | AGCGTGCCACCATCAACTGCAAGTCCAGCCAGAGTGTTTTAGACAG |
| | | CTATAACAATGAGAACAATTTAGCTTGGTATCAGCAGAAACCAGGA |
| | | CAGCCTCCTAAGCTGCTCATTTACTGGGCATCTACCCGGGAATCCG |
| | | GGGTCCCTGACCGATTCAGTGGCAGCGGGTCTGGGACAGATTTCAC |
| | | TCTCACCATCAGCAGCCTGCAGGCTGAAGATGTGGCAGTTTATTAC |
| | | TGTCAGCAATATACCAGCGAACCTATCACGTTCGGCCAAGGTACCA |
| | | AGGTGGAAATCAAAGCCGCTGCCACAACCACGCCTGCCCCTCGGCC |
| | | ACCCACGCCTGCACCCACTATCGCTTCCCAGCCACTCTCTCTTCGGC |
| | | CGGAGGCTTGTCGCCCCGCAGCGGGAGGCGCGGTTCATACTCGCGG |
| | | GCTGGACTTTGCTTGCGACATCTACATCTGGGCACCGCTTGCCGGA |
| | | ACGTGCGGGGTCTTGCTGCTGTCCCTCGTTATTACTCTTTACTGCAA |
| | | AAGAGGAAGAAAAAAGTTGCTGTATATTTTTAAGCAACCATTTATG |
| | | CGCCCGGTCCAAACTACGCAAGAGGAGGATGGATGTAGCTGCCGA |
| | | TTCCCCGAAGAAGAGGAGGGTGGTTGCGAACTGAGAGTGAAATTT |
| | | AGCCGGTCTGCTGACGCTCCGGCCTATCAGCAAGGGCAAAACCAAC |
| | | TTTACAATGAGCTTAACCTGGGGAGGCGAGAGGAATATGATGTATT |
| | | GGATAAGCGCCGAGGGAGGGACCCAGAGATGGGAGGAAAACCGA |
| | | GGAGAAAAAACCCGCAAGAGGGGCTTTATAATGAACTGCAGAAAG |
| | | ATAAGATGGCGGAGGCTTACAGCGAGATCGGGATGAAGGGAGAGA |
| | | GACGCAGAGGGAAAGGCCACGACGGTCTCTACCAAGGCCTGAGTA |
| | | CGGCCACGAAAGATACATACGATGCCCTCCATATGCAGGCCCTGCC |
| | | ACCGAGG |
| 30 | pCB7201 | ATGGCGTTGCCCGTGACCGCACTTCTGCTTCCATTGGCACTCCTGCT |
| | CAR | CCACGCGGCAAGACCGGAGGTGCAGCTGTTGGAGTCTGGGGGAGG |
| | | CTTGGTACAGCCTGGGGGGTCCCTGCGACTCTCCTGTGCAGCCTCT |
| | | GGATTCACCTTTAGCGACTATCAGATGAGCTGGGTCCGCCAGGCTC |
| | | CAGGGAAGGGGCTGGAGTGGGTGTCAGGCATTCAGGGTGGCGGTG |
| | | GTAGCACATATTACGCAGACTCCGTGAAGGGCCGGTTCACCATCTC |
| | | CCGTGACAATTCCAAGAACACGCTGTATCTGCAAATGAACAGCCTG |
| | | CGTGCCGAGGACACGGCTGTGTATTACTGTGCGAGAGAGATGTGGC |
| | | GTGGGGACTACTACTCCGGTATGGACGTCTGGGGCCAGGGGACCAC |
| | | GGTCACCGTCTCCTCAGGTGGTGGTGGTTCAGGTGGTGGTGGTTCT |
| | | GGAGGGGGCGGTTCTGGCGGCGGCTCCGGTGGTGGTGGATCCGAC |
| | | ATCGTGATGACCCAGTCTCCAGACTCCCTGGCTGTGTCTCTGGGCG |
| | | AGCGTGCCACCATCAACTGCAAGTCCAGCCAGAGTGTTTTAGACAG |
| | | CTATAACAATGAGAACAATTTAGCTTGGTATCAGCAGAAACCAGGA |
| | | CAGCCTCCTAAGCTGCTCATTTACTGGGCATCTACCCGGGAATCCG |
| | | GGGTCCCTGACCGATTCAGTGGCAGCGGGTCTGGGACAGATTTCAC |
| | | TCTCACCATCAGCAGCCTGCAGGCTGAAGATGTGGCAGTTTATTAC |
| | | TGTCAGCAATATACCAGCGAACCTATCACGTTCGGCCAAGGTACCA |
| | | AGGTGGAAATCAAAGCCGCTGCCATAGAAGTCATGTATCCTCCGCC |
| | | TTACCTGGATAACGAGAAGAGTAACGGAACTATCATACACGTGAA |
| | | GGGCAAACACCTCTGCCCCAGTCCCTTGTTTCCCGGCCCCAGCAAG |
| | | CCATTCTGGGTTTTGGTAGTGGTGGGTGGCGTCTTGGCTTGTTATTC |
| | | TCTCCTTGTCACAGTGGCATTTATTATCTTCTGGGTAAAAAGAGGA |
| | | AGAAAAAAGTTGCTGTATATTTTTAAGCAACCATTTATGCGCCCGG |
| | | TCCAAACTACGCAAGAGGAGGATGGATGTAGCTGCCGATTCCCCGA |
| | | AGAAGAGGAGGGTGGTTGCGAACTGAGAGTGAAATTTAGCCGGTC |
| | | TGCTGACGCTCCGGCCTATCAGCAAGGGCAAAACCAACTTTACAAT |
| | | GAGCTTAACCTGGGGAGGCGAGAGGAATATGATGTATTGGATAAG |
| | | CGCCGAGGGAGGGACCCAGAGATGGGAGGAAAACCGAGGAGAAA |
| | | AAACCCGCAAGAGGGGCTTTATAATGAACTGCAGAAAGATAAGAT |
| | | GGCGGAGGCTTACAGCGAGATCGGGATGAAGGGAGAGAGACGCAG |
| | | AGGGAAAGGCCACGACGGTCTCTACCAAGGCCTGAGTACGGCCAC |
| | | GAAAGATACATACGATGCCCTCCATATGCAGGCCCTGCCACCGAGG |
| 31 | pCB7204 | ATGGCGTTGCCCGTGACCGCACTTCTGCTTCCATTGGCACTCCTGCT |
| | CAR | CCACGCGGCAAGACCGGAGGTGCAGCTGTTGGAGTCTGGGGGAGG |
| | | CTTGGTACAGCCTGGGGGGTCCCTGCGACTCTCCTGTGCAGCCTCT |
| | | GGATTCACCTTTAGCGACTATCAGATGAGCTGGGTCCGCCAGGCTC |
| | | CAGGGAAGGGGCTGGAGTGGGTGTCAGGCATTCAGGGTGGCGGTG |
| | | GTAGCACATATTACGCAGACTCCGTGAAGGGCCGGTTCACCATCTC |
| | | CCGTGACAATTCCAAGAACACGCTGTATCTGCAAATGAACAGCCTG |
| | | CGTGCCGAGGACACGGCTGTGTATTACTGTGCGAGAGAGATGTGGC |
| | | GTGGGGACTACTACTCCGGTATGGACGTCTGGGGCCAGGGGACCAC |
| | | GGTCACCGTCTCCTCAGGTGGTGGTGGTTCAGGTGGTGGTGGTTCT |
| | | GGAGGGGGCGGTTCTGGCGGCGGCTCCGGTGGTGGTGGATCCGAC |
| | | ATCGTGATGACCCAGTCTCCAGACTCCCTGGCTGTGTCTCTGGGCG |
| | | AGCGTGCCACCATCAACTGCAAGTCCAGCCAGAGTGTTTTAGACAG |
| | | CTATAACAATGAGAACAATTTAGCTTGGTATCAGCAGAAACCAGGA |
| | | CAGCCTCCTAAGCTGCTCATTTACTGGGCATCTACCCGGGAATCCG |
| | | GGGTCCCTGACCGATTCAGTGGCAGCGGGTCTGGGACAGATTTCAC |
| | | TCTCACCATCAGCAGCCTGCAGGCTGAAGATGTGGCAGTTTATTAC |
| | | TGTCAGCAATATACCAGCGAACCTATCACGTTCGGCCAAGGTACCA |
| | | AGGTGGAAATCAAAGCCGCTGCCATAGAAGTCATGTATCCTCCGCC |
| | | TTACCTGGATAACGAGAAGAGTAACGGAACTATCATACACGTGAA |
| | | GGGCAAACACCTCTGCCCCAGTCCCTTGTTTCCCGGCCCCAGCAAG |
| | | CCATTCTGGGTTTTGGTAGTGGTGGGTGGCGTCTTGGCTTGTTATTC |
| | | TCTCCTTGTCACAGTGGCATTTATTATCTTCTGGGTAAAGAATCGCA |
| | | AAGCGAAAGCAAAGCCTGTAACGAGGGGGGCTGGTGCAGGGGGCA |
| | | GACAGAGGGGTCAAAATAAAGAGAGGCCGCCACCAGTACCGAACC |
| | | CTGACTATGAGCCAATCCGAAAAGGTCAACGCGATCTTTACAGCGG |
| | | GCTCAATCAGAGAAGAATACGCTCCAAACGAAGCCGACTCCTGCA |
| | | CAGTGACTACATGAATATGACACCCCGGCGGCCTGGTCCCACTAGG |
| | | AAACATTACCAGCCATATGCTCCTCCCAGGGACTTTGCCGCATATC |
| | | GGAGTAGAGTGAAATTTAGCCGGTCTGCTGACGCTCCGGCCTATCA |
| | | GCAAGGGCAAAACCAACTTTACAATGAGCTTAACCTGGGGAGGCG |
| | | AGAGGAATATGATGTATTGGATAAGCGCCGAGGGAGGGACCCAGA |
| | | GATGGGAGGAAAACCGAGGAGAAAAAACCCGCAAGAGGGGCTTTA |
| | | TAATGAACTGCAGAAAGATAAGATGGCGGAGGCTTACAGCGAGAT |
| | | CGGGATGAAGGGAGAGAGACGCAGAGGGAAAGGCCACGACGGTC |
| | | TCTACCAAGGCCTGAGTACGGCCACGAAAGATACATACGATGCCCT |
| | | CCATATGCAGGCCCTGCCACCGAGG |
| 32 | pCB7083 | ATGGCTCTCCCAGTGACTGCCCTACTGCTTCCCCTAGCGCTTCTCCT |
| | CAR | GCATGCAGAGGTGCAGCTGTTGGAGTCTGGGGGAGGCTTGGTACA |
| | | GCCTGGGGGGTCCCTGCGACTCTCCTGTGCAGCCTCTGGATTCACCT |
| | | TTAGCGACTATCAGATGAGCTGGGTCCGCCAGGCTCCAGGGAAGG |
| | | GGCTGGAGTGGGTGTCAGGCATTCAGGGTGGCGGTGGTAGCACAT |
| | | ATTACGCAGACTCCGTGAAGGGCCGGTTCACCATCTCCCGTGACAA |
| | | TTCCAAGAACACGCTGTATCTGCAAATGAACAGCCTGCGTGCCGAG |
| | | GACACGGCTGTGTATTACTGTGCGAGAGAGATGTGGCGTGGGGACT |
| | | ACTACTCCGGTATGGACGTCTGGGGCCAGGGGACCACGGTCACCGT |
| | | CTCCTCAGGCGGGGGTGGTAGTGGCGGTGGAGGTAGCGGAGGTGG |
| | | CGGGTCTGACATCGTGATGACCCAGTCTCCAGACTCCCTGGCTGTG |
| | | TCTCTGGGCGAGCGTGCCACCATCAACTGCAAGTCCAGCCAGAGTG |
| | | TTTTAGACAGCTATAACAATGAGAACAATTTAGCTTGGTATCAGCA |
| | | GAAACCAGGACAGCCTCCTAAGCTGCTCATTTACTGGGCATCTACC |
| | | CGGGAATCCGGGGTCCCTGACCGATTCAGTGGCAGCGGGTCTGGGA |
| | | CAGATTTCACTCTCACCATCAGCAGCCTGCAGGCTGAAGATGTGGC |
| | | AGTTTATTACTGTCAGCAATATACCAGCGAACCTATCACGTTCGGC |
| | | CAAGGTACCAAGGTGGAAATCAAAGAACAGAAACTGATCTCTGAA |
| | | GAAGACCTGGCGGCCGCAATTGAAGTTATGTATCCTCCTCCTTACC |
| | | TAGACAATGAGAAGAGCAATGGAACCATTATCCATGTGAAAGGGA |
| | | AACACCTTTGTCCAAGTCCCCTATTTCCCGGACCTTCTAAGCCCTTT |
| | | TGGGTGCTGGTGGTGGTTGGTGGAGTCCTGGCTTGCTATAGCTTGCT |
| | | AGTAACAGTGGCCTTTATTATTTTCTGGGTGAGGAGTAAGAGGAGC |
| | | AGGCTCCTGCACAGTGACTACATGAACATGACTCCCCGCCGCCCCG |
| | | GGCCCACCCGCAAGCATTACCAGCCCTATGCCCCACCACGCGACTT |
| | | CGCAGCCTATCGCTCCAGAGTGAAGTTCAGCAGGAGCGCAGACGC |
| | | CCCCGCGTACCAGCAGGGCCAGAACCAGCTCTATAACGAGCTCAAT |
| | | CTAGGACGAAGAGAGGAGTACGATGTTTTGGACAAGAGACGTGGC |
| | | CGGGACCCTGAGATGGGGGGAAAGCCGAGAAGGAAGAACCCTCAG |
| | | GAAGGCCTGTACAATGAACTGCAGAAAGATAAGATGGCGGAGGCC |
| | | TACAGTGAGATTGGGATGAAAGGCGAGCGCCGGAGGGGCAAGGGG |
| | | CACGATGGCCTTTACCAGGGTCTCAGTACAGCCACCAAGGACACCT |
| | | ACGACGCCCTTCACATGCAGGCCCTGCCCCCTCGC |
| 33 | pCB7084 | ATGGCTCTCCCAGTGACTGCCCTACTGCTTCCCCTAGCGCTTCTCCT |
| | CAR | GCATGCAGAGGTGCAGCTGTTGGAGTCTGGGGGAGGCTTGGTACA |
| | | GCCTGGGGGGTCCCTGCGACTCTCCTGTGCAGCCTCTGGATTCACCT |
| | | TTAGCGACTATCAGATGAGCTGGGTCCGCCAGGCTCCAGGGAAGG |
| | | GGCTGGAGTGGGTGTCAGGCATTCAGGGTGGCGGTGGTAGCACAT |
| | | ATTACGCAGACTCCGTGAAGGGCCGGTTCACCATCTCCCGTGACAA |
| | | TTCCAAGAACACGCTGTATCTGCAAATGAACAGCCTGCGTGCCGAG |
| | | GACACGGCTGTGTATTACTGTGCGAGAGAGATGTGGCGTGGGGACT |
| | | ACTACTCCGGTATGGACGTCTGGGGCCAGGGGACCACGGTCACCGT |
| | | CTCCTCAGGCGGGGGTGGTAGTGGCGGTGGAGGTAGCGGAGGTGG |
| | | CGGGTCTGACATCGTGATGACCCAGTCTCCAGACTCCCTGGCTGTG |
| | | TCTCTGGGCGAGCGTGCCACCATCAACTGCAAGTCCAGCCAGAGTG |
| | | TTTTAGACAGCTATAACAATGAGAACAATTTAGCTTGGTATCAGCA |
| | | GAAACCAGGACAGCCTCCTAAGCTGCTCATTTACTGGGCATCTACC |
| | | CGGGAATCCGGGGTCCCTGACCGATTCAGTGGCAGCGGGTCTGGGA |
| | | CAGATTTCACTCTCACCATCAGCAGCCTGCAGGCTGAAGATGTGGC |
| | | AGTTTATTACTGTCAGCAATATACCAGCGAACCTATCACGTTCGGC |
| | | CAAGGTACCAAGGTGGAAATCAAAGAACAGAAACTGATCTCTGAA |
| | | GAAGACCTGGCGGCCGCAATTGAAGTTATGTATCCTCCTCCTTACC |
| | | TAGACAATGAGAAGAGCAATGGAACCATTATCCATGTGAAAGGGA |
| | | AACACCTTTGTCCAAGTCCCCTATTTCCCGGACCTTCTAAGCCCTTT |
| | | TGGGTGCTGGTGGTGGTTGGTGGAGTCCTGGCTTGCTATAGCTTGCT |
| | | AGTAACAGTGGCCTTTATTATTTTCTGGGTGAGGAGTAAGAGGAGC |
| | | AGGCTCCTGCACAGTGACTACATGAACATGACTCCCCGCCGCCCCG |
| | | GGCCCACCCGCAAGCATTACCAGCCCTATGCCCCACCACGCGACTT |
| | | CGCAGCCTATCGCTCCAGAGTGAAGTTCAGCAGGAGCGCAGACGC |
| | | CCCCGCGTACCAGCAGGGCCAGAACCAGCTCTATAACGAGCTCAAT |
| | | CTAGGACGAAGAGAGGAGTACGATGTTTTGGACAAGAGACGTGGC |
| | | CGGGACCCTGAGATGGGGGGAAAGCCGAGAAGGAAGAACCCTCAG |
| | | GAAGGCCTGTACAATGAACTGCAGAAAGATAAGATGGCGGAGGCC |
| | | TACAGTGAGATTGGGATGAAAGGCGAGCGCCGGAGGGGCAAGGGG |
| | | CACGATGGCCTTTACCAGGGTCTCAGTACAGCCACCAAGGACACCT |
| | | ACGACGCCCTTCACATGCAGGCCCTGCCCCCTCGC |
| 34 | pCB7085 | ATGGCTCTCCCAGTGACTGCCCTACTGCTTCCCCTAGCGCTTCTCCT |
| | CAR | GCATGCAGAGGTGCAGCTGTTGGAGTCTGGGGGAGGCTTGGTACA |
| | | GCCTGGGGGGTCCCTGCGACTCTCCTGTGCAGCCTCTGGATTCACCT |
| | | TTAGCGACTATCAGATGAGCTGGGTCCGCCAGGCTCCAGGGAAGG |
| | | GGCTGGAGTGGGTGTCAGGCATTCAGGGTGGCGGTGGTAGCACAT |
| | | ATTACGCAGACTCCGTGAAGGGCCGGTTCACCATCTCCCGTGACAA |
| | | TTCCAAGAACACGCTGTATCTGCAAATGAACAGCCTGCGTGCCGAG |
| | | GACACGGCTGTGTATTACTGTGCGAGAGAGATGTGGCGTGGGGACT |
| | | ACTACTCCGGTATGGACGTCTGGGGCCAGGGGACCACGGTCACCGT |
| | | CTCCTCAGGTGGTGGTGGTTCAGGTGGTGGTGGTTCTGGAGGGGGC |
| | | GGTTCTGGCGGCGGCTCCGGTGGTGGTGGATCCGACATCGTGATGA |
| | | CCCAGTCTCCAGACTCCCTGGCTGTGTCTCTGGGCGAGCGTGCCAC |
| | | CATCAACTGCAAGTCCAGCCAGAGTGTTTTAGACAGCTATAACAAT |
| | | GAGAACAATTTAGCTTGGTATCAGCAGAAACCAGGACAGCCTCCTA |
| | | AGCTGCTCATTTACTGGGCATCTACCCGGGAATCCGGGGTCCCTGA |
| | | CCGATTCAGTGGCAGCGGGTCTGGGACAGATTTCACTCTCACCATC |
| | | AGCAGCCTGCAGGCTGAAGATGTGGCAGTTTATTACTGTCAGCAAT |
| | | ATACCAGCGAACCTATCACGTTCGGCCAAGGTACCAAGGTGGAAAT |
| | | CAAAGAACAGAAACTGATCTCTGAAGAAGACCTGGCGGCCGCAAT |
| | | TGAAGTTATGTATCCTCCTCCTTACCTAGACAATGAGAAGAGCAAT |
| | | GGAACCATTATCCATGTGAAAGGGAAACACCTTTGTCCAAGTCCCC |
| | | TATTTCCCGGACCTTCTAAGCCCTTTTGGGTGCTGGTGGTGGTTGGT |
| | | GGAGTCCTGGCTTGCTATAGCTTGCTAGTAACAGTGGCCTTTATTAT |
| | | TTTCTGGGTGAGGAGTAAGAGGAGCAGGCTCCTGCACAGTGACTAC |
| | | ATGAACATGACTCCCCGCCGCCCCGGGCCCACCCGCAAGCATTACC |
| | | AGCCCTATGCCCCACCACGCGACTTCGCAGCCTATCGCTCCAGAGT |
| | | GAAGTTCAGCAGGAGCGCAGACGCCCCCGCGTACCAGCAGGGCCA |
| | | GAACCAGCTCTATAACGAGCTCAATCTAGGACGAAGAGAGGAGTA |
| | | CGATGTTTTGGACAAGAGACGTGGCCGGGACCCTGAGATGGGGGG |
| | | AAAGCCGAGAAGGAAGAACCCTCAGGAAGGCCTGTACAATGAACT |
| | | GCAGAAAGATAAGATGGCGGAGGCCTACAGTGAGATTGGGATGAA |
| | | AGGCGAGCGCCGGAGGGGCAAGGGGCACGATGGCCTTTACCAGGG |
| | | TCTCAGTACAGCCACCAAGGACACCTACGACGCCCTTCACATGCAG |
| | | GCCCTGCCCCCTCGC |
| 35 | pCB7086 | ATGGCTCTCCCAGTGACTGCCCTACTGCTTCCCCTAGCGCTTCTCCT |
| | CAR | GCATGCAGACATCGTGATGACCCAGTCTCCAGACTCCCTGGCTGTG |
| | | TCTCTGGGCGAGCGTGCCACCATCAACTGCAAGTCCAGCCAGAGTG |
| | | TTTTAGACAGCTATAACAATGAGAACAATTTAGCTTGGTATCAGCA |
| | | GAAACCAGGACAGCCTCCTAAGCTGCTCATTTACTGGGCATCTACC |
| | | CGGGAATCCGGGGTCCCTGACCGATTCAGTGGCAGCGGGTCTGGGA |
| | | CAGATTTCACTCTCACCATCAGCAGCCTGCAGGCTGAAGATGTGGC |
| | | AGTTTATTACTGTCAGCAATATACCAGCGAACCTATCACGTTCGGC |
| | | CAAGGTACCAAGGTGGAAATCAAAGGTGGTGGTGGTTCAGGTGGT |
| | | GGTGGTTCTGGCGGCGGCTCCGGTGGTGGTGGATCCGAGGTGCAGC |
| | | TGTTGGAGTCTGGGGGAGGCTTGGTACAGCCTGGGGGGTCCCTGCG |
| | | ACTCTCCTGTGCAGCCTCTGGATTCACCTTTAGCGACTATCAGATGA |
| | | GCTGGGTCCGCCAGGCTCCAGGGAAGGGGCTGGAGTGGGTGTCAG |
| | | GCATTCAGGGTGGCGGTGGTAGCACATATTACGCAGACTCCGTGAA |
| | | GGGCCGGTTCACCATCTCCCGTGACAATTCCAAGAACACGCTGTAT |
| | | CTGCAAATGAACAGCCTGCGTGCCGAGGACACGGCTGTGTATTACT |
| | | GTGCGAGAGAGATGTGGCGTGGGGACTACTACTCCGGTATGGACGT |
| | | CTGGGGCCAGGGGACCACGGTCACCGTCTCCTCAGAACAGAAACT |
| | | GATCTCTGAAGAAGACCTGGCGGCCGCAATTGAAGTTATGTATCCT |
| | | CCTCCTTACCTAGACAATGAGAAGAGCAATGGAACCATTATCCATG |
| | | TGAAAGGGAAACACCTTTGTCCAAGTCCCCTATTTCCCGGACCTTCT |
| | | AAGCCCTTTTGGGTGCTGGTGGTGGTTGGTGGAGTCCTGGCTTGCT |
| | | ATAGCTTGCTAGTAACAGTGGCCTTTATTATTTTCTGGGTGAGGAGT |
| | | AAGAGGAGCAGGCTCCTGCACAGTGACTACATGAACATGACTCCCC |
| | | GCCGCCCCGGGCCCACCCGCAAGCATTACCAGCCCTATGCCCCACC |
| | | ACGCGACTTCGCAGCCTATCGCTCCAGAGTGAAGTTCAGCAGGAGC |
| | | GCAGACGCCCCCGCGTACCAGCAGGGCCAGAACCAGCTCTATAAC |
| | | GAGCTCAATCTAGGACGAAGAGAGGAGTACGATGTTTTGGACAAG |
| | | AGACGTGGCCGGGACCCTGAGATGGGGGGAAAGCCGAGAAGGAA |
| | | GAACCCTCAGGAAGGCCTGTACAATGAACTGCAGAAAGATAAGAT |
| | | GGCGGAGGCCTACAGTGAGATTGGGATGAAAGGCGAGCGCCGGAG |
| | | GGGCAAGGGGCACGATGGCCTTTACCAGGGTCTCAGTACAGCCACC |
| | | AAGGACACCTACGACGCCCTTCACATGCAGGCCCTGCCCCCTCGC |
| 36 | pCB7087 | ATGGCTCTCCCAGTGACTGCCCTACTGCTTCCCCTAGCGCTTCTCCT |
| | CAR | GCATGCAGAGGTGCAGCTGTTGGAGTCTGGGGGAGGCTTGGTACA |
| | | GCCTGGGGGGTCCCTGCGACTCTCCTGTGCAGCCTCTGGATTCACCT |
| | | TTACCAGCTATGCCATGAGCTGGGTCCGCCAGGCTCCAGGGAAGGG |
| | | GCTGGAGTGGGTGTCAGGCATTGACGGTAGCGGTGGTGGCACAAA |
| | | TTACGCAGACTCCGTGAAGGGCCGGTTCACCATCTCCCGTGACAAT |
| | | TCCAAGAACACGCTGTATCTGCAAATGAACAGCCTGCGTGCCGAGG |
| | | ACACGGCTGTGTATTACTGTGCGAGAGCGTATTACGATATTTTGAC |
| | | TGGTTACCCCGTGGACGGTATGGACGTCTGGGGCCAAGGGACCACG |
| | | GTCACCGTCTCCTCAGGCGGGGGTGGTAGTGGCGGTGGAGGTAGCG |
| | | GAGGTGGCGGGTCTGACATCGTGATGACCCAGTCTCCAGACTCCCT |
| | | GGCTGTGTCTCTGGGCGAGCGTGCCACCATCAACTGCAAGTCCAGC |
| | | CAGAGTGTTTTAAGCAGCTATAACAATGAGAACAATTTAGCTTGGT |
| | | ATCAGCAGAAACCAGGACAGCCTCCTAAGCTGCTCATTTACGCCGC |
| | | ATCTACCCGGGAATCCGGGGTCCCTGACCGATTCAGTGGCAGCGGG |
| | | TCTGGGACAGATTTCACTCTCACCATCAGCAGCCTGCAGGCTGAAG |
| | | ATGTGGCAGTTTATTACTGTCAGCAATATTATAGCGAACCTTATAC |
| | | GTTCGGCCAAGGTACCAAGGTGGAAATCAAAGAACAGAAACTGAT |
| | | CTCTGAAGAAGACCTGGCGGCCGCAATTGAAGTTATGTATCCTCCT |
| | | CCTTACCTAGACAATGAGAAGAGCAATGGAACCATTATCCATGTGA |
| | | AAGGGAAACACCTTTGTCCAAGTCCCCTATTTCCCGGACCTTCTAA |
| | | GCCCTTTTGGGTGCTGGTGGTGGTTGGTGGAGTCCTGGCTTGCTATA |
| | | GCTTGCTAGTAACAGTGGCCTTTATTATTTTCTGGGTGAGGAGTAA |
| | | GAGGAGCAGGCTCCTGCACAGTGACTACATGAACATGACTCCCCGC |
| | | CGCCCCGGGCCCACCCGCAAGCATTACCAGCCCTATGCCCCACCAC |
| | | GCGACTTCGCAGCCTATCGCTCCAGAGTGAAGTTCAGCAGGAGCGC |
| | | AGACGCCCCCGCGTACCAGCAGGGCCAGAACCAGCTCTATAACGA |
| | | GCTCAATCTAGGACGAAGAGAGGAGTACGATGTTTTGGACAAGAG |
| | | ACGTGGCCGGGACCCTGAGATGGGGGGAAAGCCGAGAAGGAAGA |
| | | ACCCTCAGGAAGGCCTGTACAATGAACTGCAGAAAGATAAGATGG |
| | | CGGAGGCCTACAGTGAGATTGGGATGAAAGGCGAGCGCCGGAGGG |
| | | GCAAGGGGCACGATGGCCTTTACCAGGGTCTCAGTACAGCCACCAA |
| | | GGACACCTACGACGCCCTTCACATGCAGGCCCTGCCCCCTCGC |
| 37 | TRAC | TrAArUrUrUCrUrACrUCrUTGrUrArGArUGrArGrUrCrUrCrUrCrAGrCrUr |
| | chRDNA | GrGrUrArCrAC |
| | targeting | (rN is a ribonucleotide; N is a deoxyribonucleotide) |
| 38 | B2M | TrAArUrUrUCrUrACrUCrUTGrUrArGArUArGrUrGrGrGrGrGrUrGArArUr |
| | chRDNA | UrCrArGrUrGT |
| | targeting | (rN is a ribonucleotide; N is a deoxyribonucleotide) |
| 39 | PDCD1 | rUrAArUrUrUrCrUrArCrUrCrUTGrUrArGrArUGrCrArCrGrArAGrCrUrCrU |
| | chRDNA | rCrCrGrArUrGrUrG |
| | targeting | (rN is a ribonucleotide; N is a deoxyribonucleotide) |
| 40 | B2M- | TGAGTGCTGAGAGGGCATCAGAAGTCCTTGAGAGCCTCCAGAGAA |
| | HLA-E | AGGCTCTTAAAAATGCAGCGCAATCTCCAGTGACAGAAGATACTGC |
| | | TAGAAATCTGCTAGAAAAAAAACAAAAAAGGCATGTATAGAGGAA |
| | | TTATGAGGGAAAGATACCAAGTCACGGTTTATTCTTCAAAATGGAG |
| | | GTGGCTTGTTGGGAAGGTGGAAGCTCATTTGGCCAGAGTGGAAATG |
| | | GAATTGGGAGAAATCGATGACCAAATGTAAACACTTGGTGCCTGAT |
| | | ATAGCTTGACACCAAGTTAGCCCCAAGTGAAATACCCTGGCAATAT |
| | | TAATGTGTCTTTTCCCGATATTCCTCAGGTACTCCAAAGATTCAGGT |
| | | TTACTCACGTCATCCAGCAGAGAATGGAAAGTCAAATTTCCTGAAT |
| | | TGCTATGTGTCTGGGTTTCATCCATCCGACATTGAAGTTGACTTACT |
| | | GAAGAATGGAGAGAGAATTGAAAAAGTGGAGCATTCAGACTTGTC |
| | | TTTCAGCAAGGACTGGTCTTTCTATCTCTTGTACTACACTGAATTTG |
| | | GTTCTGGAGCAACCAACTTCTCCTTGCTGAAACAGGCCGGTGACGT |
| | | GGAGGAGAATCCCGGCCCTATGAGCAGAAGTGTCGCCTTAGCTGTG |
| | | CTCGCGCTACTCTCTCTTTCTGGCCTGGAGGCTGTAATGGCACCACG |
| | | GACCCTCTTTCTGGGTGGAGGTGGGAGCGGAGGAGGCGGTAGCGG |
| | | AGGCGGCGGCTCCATCCAGCGTACTCCAAAGATTCAGGTTTACTCA |
| | | CGTCATCCAGCAGAGAATGGAAAGTCAAATTTCCTGAATTGCTATG |
| | | TGTCTGGGTTTCATCCATCCGACATTGAAGTTGACTTACTGAAGAAT |
| | | GGAGAGAGAATTGAAAAAGTGGAGCATTCAGACTTGTCTTTCAGCA |
| | | AGGACTGGTCTTTCTATCTCTTGTACTATACAGAGTTTACACCTACG |
| | | AGAAAGATGAGTATGCCTGCCGTGTGAACCATGTGACTTTGTCACA |
| | | GCCCAAGATCGTGAAATGGGATCGAGACATGGGTGGCGGAGGGTC |
| | | TGGCGGAGGCGGTTCTGGAGGAGGGGGATCTGGTGGCGGAGGGTC |
| | | TGGATCCCACTCCTTGAAGTATTTCCACACTTCCGTGTCCCGGCCCG |
| | | GCCGCGGGGAGCCCCGCTTCATCTCTGTGGGCTACGTGGACGACAC |
| | | CCAGTTCGTGCGCTTCGACAACGACGCCGCGAGTCCGAGGATGGTG |
| | | CCGCGGGCGCCGTGGATGGAGCAGGAGGGGTCAGAGTATTGGGAC |
| | | CGGGAGACACGGAGCGCCAGGGACACCGCACAGATTTTCCGAGTG |
| | | AATCTGCGGACGCTGCGCGGCTACTACAATCAGAGCGAGGCCGGG |
| | | TCTCACACCCTGCAGTGGATGCATGGCTGCGAGCTGGGGCCCGACG |
| | | GGCGCTTCCTCCGCGGGTATGAACAGTTCGCCTACGACGGCAAGGA |
| | | TTATCTCACCCTGAATGAGGACCTGCGCTCCTGGACCGCGGTGGAC |
| | | ACGGCGGCTCAGATCTCCGAGCAAAAGTCAAATGATGCTTCTGAGG |
| | | CGGAGCACCAGAGAGCCTACCTGGAAGACACATGCGTGGAGTGGC |
| | | TCCACAAATACCTGGAGAAGGGGAAGGAGACGCTGCTTCACCTGG |
| | | AGCCCCCAAAGACACACGTGACTCACCACCCCATCTCTGACCATGA |
| | | GGCCACCCTGAGGTGCTGGGCCCTGGGCTTCTACCCTGCGGAGATC |
| | | ACACTGACCTGGCAGCAGGATGGGGAGGGCCATACCCAGGACACG |
| | | GAGCTCGTGGAGACCAGGCCTGCAGGGGATGGAACCTTCCAGAAG |
| | | TGGGCAGCTGTGGTGGTGCCTTCTGGAGAGGAGCAGAGATACACG |
| | | TGCCATGTGCAGCATGAGGGGCTACCCGAGCCCGTCACCCTGAGAT |
| | | GGAAGCCGGCTTCCCAGCCCACCATCCCCATCGTGGGCATCATTGC |
| | | TGGCCTGGTTCTCCTTGGATCTGTGGTCTCTGGAGCTGTGGTTGCTG |
| | | CTGTGATATGGAGGAAGAAGAGCTCAGGTGGAAAAGGAGGGAGCT |
| | | ACTCTAAGGCTGAGTGGAGCGACAGTGCCCAGGGGTCTGAGTCTCA |
| | | CAGCTTGTAATAACTGTGCC TTCTAGTTGC |
| 41 | chRDNA | GrArGrUrCrUrCrUrCrAGrCrUrGrGrUrArCrAC |
| | backbone | (rN is a ribonucleotide; N is a deoxyribonucleotide) |

1. An immune cell comprising a chimeric antigen receptor (CAR) comprising:
(i) an anti-CLL-1 scFv;
(ii) a hinge domain;
(iii) a CD8 transmembrane domain;
(iv) a CD28 co-stimulatory domain; and
(v) a CD3 zeta domain,
the immune cell further comprising armoring genomic modifications comprising inactivation of the PDCD1 gene and inactivation of the beta-2 microglobulin (B2M) gene.

2. (canceled)

3. The immune cell of claim 1, wherein the anti-CLL-1 scFv is represented by a formula V_{H}-Lₙ-V_{L}, wherein V_{H} comprises SEQ ID NO: 7, V_{L} comprises SEQ ID NO: 11, and Lₙ is a peptide linker having the sequence
GGGGSGGGGSGGGGSGGGSGGGGS.

4-6. (canceled)

7. The immune cell of claim 3, wherein the anti-CLL-1 scFv comprises or consists essentially of SEQ ID NO: 5.

8-18. (canceled)

19. The immune cell of claim 1, wherein the hinge domain comprises a CD8 hinge domain consisting essentially of SEQ ID NO. 15.

20. (canceled)

21. The immune cell of claim 1, wherein the hinge domain comprises or consists essentially of a CD28 hinge domain.

22-24. (canceled)

25. The immune cell of claim 1, wherein the CAR comprises or consists essentially of SEQ ID NO: 22 without the CD28 signal peptide
MALPVTALLLPLALLLHAARP.

26-40. (canceled)

41. The immune cell of claim 1, wherein the chimeric antigen receptor (CAR) is inserted into the T-cell receptor alpha chain (TRAC) locus on human chromosome 14 between nucleotides 22547538 and 22547539.

42-45. (canceled)

46. The immune cell of claim 1, wherein the PDCD1 gene is cleaved between nucleotides 241852860 and 241852883.

47. (canceled)

48. The immune cell of claim 1, wherein the armoring genomic modification further comprises insertion of an HLA-E-B2M fusion coding sequence into the B2Mlocus on human chromosome 15 between nucleotides 44715624 and 44715625.

49-51. (canceled)

52. A method of making the immune cell of claim 1, the method comprising introducing into a cell a nucleic acid comprising SEQ ID NO: 27, and a nucleic acid encoding SEQ ID NO.: 40 and further comprising disrupting the PDCD1 gene, the TRAC gene and the B2M gene in the cell.

53. (canceled)

54. The method of claim 52, wherein the introducing step comprises introducing into the cell a sequence-dependent endonuclease.

55. The method of claim 54, wherein the introducing step comprises introducing into the cell a CRISPR Cas12a system comprising a nucleic acid-guided endonuclease and nucleic acid-targeting nucleic acid (NATNA).

56-62. (canceled)

63. The method of claim 55, wherein the endonuclease cleaves the TRAC locus between nucleotides 22547538 and 22547539 and the endonuclease forms a nucleoprotein complex with a NATNA comprising a targeting region having SEQ ID NO.: 37.

64-65. (canceled)

66. The method of claim 52, wherein SEQ ID NO: 27 is inserted into the cleaved TRAC locus.

67. The method of claim 55, wherein the endonuclease cleaves the B2Mlocus on human chromosome 15 between nucleotides 44715624 and 44715625 and the endonuclease forms a nucleoprotein complex with a NATNA comprising a targeting region having SEQ ID NO.: 38.

68-69. (canceled)

70. The method of claim 67, wherein a sequence encoding the HLA-E-B2M fusion of SEQ ID NO.: 40 is inserted into the cleaved B2Mlocus.

71-75. (canceled)

76. The method of claim 52, wherein the disrupting of the PDCD1 gene comprises introducing into the cell a CRISPR Casl2a endonuclease and NATNA comprising SEQ ID NO.: 39 wherein the endonuclease cleaves the PDCD1 locus on human chromosome 2 between nucleotides 241852860 and 241852883.

77. (canceled)

78. The immune cell of claim 1 present in a pharmaceutically acceptable excipient.

79-84. (canceled)

85. A method of inhibiting the growth of a CLL-1 expressing tumor selected from acute myeloblastic leukemia (AML) and myelodysplastic syndrome (MDS) in a patient comprising administering to a patient having the tumor the immune cell of claim 78.

86-110. (canceled)

111. The method of claim 85, the method further comprising measuring expression of CLL-1 in the cells of the tumor and administering the treatment if CLL-1 expression is detected and not administering the treatment if the CLL-1 expression is not detected.

112-117. (canceled)


SEQUENCE LISTING (44 sequences)

SEQ ID NO: 1 - protein, 4 aa, synthetic construct
GGGS

SEQ ID NO: 2 - protein, 5 aa, synthetic construct
GGGGS

SEQ ID NO: 3 - protein, 250 aa, synthetic construct
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 4 - protein, 254 aa, synthetic construct
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 5 - protein, 259 aa, synthetic construct
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 6 - protein, 254 aa, synthetic construct
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 7 - protein, 122 aa, synthetic construct
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 8 - protein, 8 aa, synthetic construct
GFTFSDYQ

SEQ ID NO: 9 - protein, 7 aa, synthetic construct
QGGGGST

SEQ ID NO: 10 - protein, 15 aa, synthetic construct
AREMWRGDYYSGMDV

SEQ ID NO: 11 - protein, 113 aa, synthetic construct
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 12 - protein, 12 aa, synthetic construct
QSVLDSYNNENN

SEQ ID NO: 13 - skipped

SEQ ID NO: 14 - protein, 9 aa, synthetic construct
QQYTSEPIT

SEQ ID NO: 15 - protein, 39 aa, synthetic construct
IEVMYPPPYLDNEKSNGTIIHVKGKHLCPSPLFPGPSKP

SEQ ID NO: 16 - protein, 27 aa, synthetic construct
FWVLVVVGGVLACYSLLVTVAFIIFWV

SEQ ID NO: 17 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 493 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 496 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 509 aa, synthetic construct
MALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 497 aa, synthetic construct
MALPVTALLLPLALLLHAARPDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 502 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 696 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 24 - protein, 506 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 503 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 557 aa, synthetic construct
MALPVTALLLPLALLLHAARPEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKAAAIEVMYPPPYLDNEKSNGTIIHVKGKHLCPSPLFPGPSKPFWVLVVVGGVLACYSLLVTVAFIIFWVKNRKAKAKPVTRGAGAGGRQRGQNKERPPPVPNPDYEPIRKGQRDLYSGLNQRRIRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1506 bp, synthetic construct
  source 1..1506: mol_type = other DNA
atggcgttgcccgtgaccgcacttctgcttccattggcactcctgctccacgcggcaagaccggaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagccgctgccatagaagtcatgtatcctccgccttacctggataacgagaagagtaacggaactatcatacacgtgaagggcaaacacctctgccccagtcccttgtttcccggccccagcaagccattctgggttttggtagtggtgggtggcgtcttggcttgttattctctccttgtcacagtggcatttattatcttctgggtacgctccaaacgaagccgactcctgcacagtgactacatgaatatgacaccccggcggcctggtcccactaggaaacattaccagccatatgctcctcccagggactttgccgcatatcggagtagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 28 - DNA, 2088 bp, synthetic construct
  source 1..2088: mol_type = other DNA
atggcgttgcccgtgaccgcacttctgcttccattggcactcctgctccacgcggcaagaccggaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagccgctgccatagaagtcatgtatcctccgccttacctggataacgagaagagtaacggaactatcatacacgtgaagggcaaacacctctgccccagtcccttgtttcccggccccagcaagccattctgggttttggtagtggtgggtggcgtcttggcttgttattctctccttgtcacagtggcatttattatcttctgggtacgctccaaacgaagccgactcctgcacagtgactacatgaatatgacaccccggcggcctggtcccactaggaaacattaccagccatatgctcctcccagggactttgccgcatatcggagtagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgaggggttctggagcaaccaacttctccttgctgaaacaggccggtgacgtggaggagaatcccggccctatgtaccgcatgcaacttctgtcttgtattgcgctttctctcgcactcgtaaccaattccagcatgtgtaagccgataaccgggactataaacgatttgaatcagcaggtatggaccttgcagggacagaatctggtagctgtaccacggagcgacagcgtcacccccgtgactgtggccgtcataacatgcaaatacccggaagcgctcgaacagggcaggggtgatccgatatacctgggaatccaaaacccggagatgtgcctttactgcgagaaggtgggcgaacaacctacgcttcaattgaaagagcaaaagataatggacctgtacggccaacccgaacccgtaaagcccttcctcttttaccgcgccaagactggtagaacaagtactctggagagcgttgcttttcctgattggtttatagcaagctccaaaagggaccagccgattatcctcacaagcgaactcggaaagtcctataataccgcttttgagttgaatatcaatgac

SEQ ID NO: 29 - DNA, 1518 bp, synthetic construct
  source 1..1518: mol_type = other DNA
atggcgttgcccgtgaccgcacttctgcttccattggcactcctgctccacgcggcaagaccggaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagccgctgccacaaccacgcctgcccctcggccacccacgcctgcacccactatcgcttcccagccactctctcttcggccggaggcttgtcgccccgcagcgggaggcgcggttcatactcgcgggctggactttgcttgcgacatctacatctgggcaccgcttgccggaacgtgcggggtcttgctgctgtccctcgttattactctttactgcaaaagaggaagaaaaaagttgctgtatatttttaagcaaccatttatgcgcccggtccaaactacgcaagaggaggatggatgtagctgccgattccccgaagaagaggagggtggttgcgaactgagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 30 - DNA, 1509 bp, synthetic construct
  source 1..1509: mol_type = other DNA
atggcgttgcccgtgaccgcacttctgcttccattggcactcctgctccacgcggcaagaccggaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagccgctgccatagaagtcatgtatcctccgccttacctggataacgagaagagtaacggaactatcatacacgtgaagggcaaacacctctgccccagtcccttgtttcccggccccagcaagccattctgggttttggtagtggtgggtggcgtcttggcttgttattctctccttgtcacagtggcatttattatcttctgggtaaaaagaggaagaaaaaagttgctgtatatttttaagcaaccatttatgcgcccggtccaaactacgcaagaggaggatggatgtagctgccgattccccgaagaagaggagggtggttgcgaactgagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 31 - DNA, 1671 bp, synthetic construct
  source 1..1671: mol_type = other DNA
atggcgttgcccgtgaccgcacttctgcttccattggcactcctgctccacgcggcaagaccggaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagccgctgccatagaagtcatgtatcctccgccttacctggataacgagaagagtaacggaactatcatacacgtgaagggcaaacacctctgccccagtcccttgtttcccggccccagcaagccattctgggttttggtagtggtgggtggcgtcttggcttgttattctctccttgtcacagtggcatttattatcttctgggtaaagaatcgcaaagcgaaagcaaagcctgtaacgaggggggctggtgcagggggcagacagaggggtcaaaataaagagaggccgccaccagtaccgaaccctgactatgagccaatccgaaaaggtcaacgcgatctttacagcgggctcaatcagagaagaatacgctccaaacgaagccgactcctgcacagtgactacatgaatatgacaccccggcggcctggtcccactaggaaacattaccagccatatgctcctcccagggactttgccgcatatcggagtagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 32 - DNA, 1500 bp, synthetic construct
  source 1..1500: mol_type = other DNA
atggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggcgggggtggtagtggcggtggaggtagcggaggtggcgggtctgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - DNA, 1500 bp, synthetic construct; identical to SEQ ID NO: 32
  source 1..1500: mol_type = other DNA

SEQ ID NO: 34 - DNA, 1527 bp, synthetic construct
  source 1..1527: mol_type = other DNA
atggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - DNA, 1512 bp, synthetic construct
  source 1..1512: mol_type = other DNA
atggctctcccagtgactgccctactgcttcccctagcgcttctcctgcatgcagacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - DNA, 1509 bp, synthetic construct
  source 1..1509: mol_type = other DNA
atggctctcccagtgactgccctactgcttcccctagcgcttctcctgcatgcagaggtgcagctgttggagtctgggggaggcttggtacagcctggggggtccctgcgactctcctgtgcagcctctggattcacctttaccagctatgccatgagctgggtccgccaggctccagggaaggggctggagtgggtgtcaggcattgacggtagcggtggtggcacaaattacgcagactccgtgaagggccggttcaccatctcccgtgacaattccaagaacacgctgtatctgcaaatgaacagcctgcgtgccgaggacacggctgtgtattactgtgcgagagcgtattacgatattttgactggttaccccgtggacggtatggacgtctggggccaagggaccacggtcaccgtctcctcaggcgggggtggtagtggcggtggaggtagcggaggtggcgggtctgacatcgtgatgacccagtctccagactccctggctgtgtctctgggcgagcgtgccaccatcaactgcaagtccagccagagtgttttaagcagctataacaatgagaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatagcgaaccttatacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA
taatttctactcttgtagatgagtctctcagctggtacac

SEQ ID NO: 38 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA
taatttctactcttgtagatagtgggggtgaattcagtgt

SEQ ID NO: 39 - DNA, 40 bp, synthetic construct
  source 1..40: mol_type = other DNA
  misc_feature 1..2: note = RNA
  misc_feature 3: note = DNA
  misc_feature 4..13: note = RNA
  misc_feature 14..15: note = DNA
  misc_feature 16..20: note = RNA
  misc_feature 21: note = DNA
  misc_feature 22..27: note = RNA
  misc_feature 28: note = DNA
  misc_feature 29..40: note = RNA
taatttctactcttgtagatgcacgaagctctccgatgtg

SEQ ID NO: 40 - DNA, 2139 bp, synthetic construct
  source 1..2139: mol_type = other DNA
tgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaatttggttctggagcaaccaacttctccttgctgaaacaggccggtgacgtggaggagaatcccggccctatgagcagaagtgtcgccttagctgtgctcgcgctactctctctttctggcctggaggctgtaatggcaccacggaccctctttctgggtggaggtgggagcggaggaggcggtagcggaggcggcggctccatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactatacagagtttacacctacgagaaagatgagtatgcctgccgtgtgaaccatgtgactttgtcacagcccaagatcgtgaaatgggatcgagacatgggtggcggagggtctggcggaggcggttctggaggagggggatctggtggcggagggtctggatcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcttctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaataactgtgccttctagttgc

SEQ ID NO: 41 - DNA, 20 bp, synthetic construct
  source 1..20: mol_type = other DNA
  misc_feature 1: note = DNA
  misc_feature 2..10: note = RNA
  misc_feature 11: note = DNA
  misc_feature 12..19: note = RNA
  misc_feature 20: note = DNA
gagtctctcagctggtacac

SEQ ID NO: 42 - protein, 50 aa, synthetic construct
  VARIANT 1..5: note = This region may encompass 1-5 residues
  VARIANT 6..10: note = This region may encompass 1-5 residues
  VARIANT 11..15: note = This region may encompass 1-5 residues
  VARIANT 16..20: note = This region may encompass 1-5 residues
  VARIANT 21..25: note = This region may encompass 1-5 residues
  VARIANT 26..30: note = This region may encompass 1-5 residues
  VARIANT 31..35: note = This region may encompass 1-5 residues
  VARIANT 36..40: note = This region may encompass 1-5 residues
  VARIANT 41..45: note = This region may encompass 1-5 residues
  VARIANT 46..50: note = This region may encompass 1-5 residues
  VARIANT 1..50: note = This sequence may encompass 1-5 GxSy repeating units, where x=1-5 and y=1-5
GGGGGSSSSSGGGGGSSSSSGGGGGSSSSSGGGGGSSSSSGGGGGSSSSS

SEQ ID NO: 43 - protein, 20 aa, synthetic construct
  VARIANT 1..20: note = This sequence may encompass 1-5 GGGS repeating units
GGGSGGGSGGGSGGGSGGGS

SEQ ID NO: 44 - protein, 25 aa, synthetic construct
  VARIANT 1..25: note = This sequence may encompass 1-5 GGGGS repeating units
GGGGSGGGGSGGGGSGGGGSGGGGS